(19) United States
(12) Patent Application Publication
(10) Pub. No.: US 2012/0114640 A1
(43) Pub. Date: May 10, 2012

(54) MODULATION OF THE TGF- ß AND PI3K/AKT PATHWAYS IN THE DIAGNOSIS AND TREATMENT OF SQUAMOUS CELL CARCINOMA

(76) Inventors: Ashok B. Kulkarni, North Potomac, MD (US); Bian Yansong, Potomac, MD (US)

(21) Appl. No.: 13/318,219

(22) PCT Filed: May 4, 2010
(86) PCT No.: PCT/US2010/033495

§ 371 Date: Oct. 31, 2011

Related U.S. Application Data
(60) Provisional application No. 61/176,723, filed on May 8, 2009.

(51) Int. Cl. A61K 39/395; C12Q 1/68; A61N 5/00; A61P 35/00; A61P 35/04; G01N 33/574; C40B 30/04

(52) U.S. Cl. 424/133.1; 435/7.23; 435/6.12; 435/6.11; 506/9; 600/1

(57) ABSTRACT

Described herein is the finding that the PI3K/Akt and TGF-β pathways act cooperatively to promote squamous cell carcinoma (SCC), such as head and neck squamous cell carcinoma (HNSCC). In particular, it was found that conditional deletion of transforming growth factor-β receptor type I (TGFBR1) and phosphatase and tensin homolog (PTEN) in head and neck epithelia of mice led to spontaneous development of SCC in the mice with complete penetrance. Accordingly, provided herein are methods of treating a subject diagnosed with SCC by administering to the subject a therapeutically effective amount of an inhibitor of the PI3K/Akt pathway and a therapeutically effective amount of a modulator of the TGF-β pathway. Also provided is a method of diagnosing a subject as having SCC, or being susceptible to developing SCC, by detecting the presence or absence of at least one tumor-associated mutation in the TGFBR1 gene and at least one tumor-associated mutation in the PTEN gene. Further provided is a method of diagnosing a subject as having SCC, or being susceptible to developing SCC, by detecting expression of TGFBR1 and PTEN in a sample obtained from the subject. Pharmaceutical compositions that include an inhibitor of the PI3K/Akt pathway and a modulator of the TGF-β pathway, and the use of such pharmaceutical compositions for the treatment of SCC, are also provided herein.

### CROSS REFERENCE TO RELATED APPLICATIONS

This application claims the benefit of U.S. Provisional Application No. 61/176,723, filed on May 8, 2009, which is herein incorporated by reference in its entirety.

### FIELD

This disclosure concerns the role of the transforming growth factor (TGF)-13 receptor type I (TGFBR1) and phosphatase and tensin homolog (PTEN) in squamous cell carcinoma (SCC). In particular, this disclosure relates to the use of modulators of the TGF-β and PI3K/Akt pathways for the treatment of SCC, and the use of TGFBR1 and PTEN as biomarkers of SCC, such as head and neck squamous cell carcinoma (HNSCC).

### BACKGROUND

Head and neck squamous cell carcinoma (HNSCC) is one of the most common types of human cancer with an annual incidence of more than 500,000 cases worldwide. In the United States alone, about 47,000 new patients are diagnosed with HNSCC each year (Siegel et al., CA Cancer J Clin 58:71-96, 2008). Despite an improvement in early diagnosis and comprehensive treatment, the overall 5-year survival rate of HNSCC patients is only about 50%, and this number has not changed in more than three decades. Tobacco, alcohol consumption and viral agents are the major risk factors for development of HNSCC. These risk factors, together with genetic susceptibility, result in the accumulation of multiple genetic and epigenetic alterations in a multistep process of cancer development (Kim and Califano, Int J Cancer 112:545-53, 2004). However, the underlying cellular and molecular mechanisms that contribute to the initiation and progression from normal epithelia to invasive squamous cell carcinoma have not been clearly delineated (Mao et al., Cancer Cell 5:311-6, 2004). A better understanding of molecular carcinogenesis of HNSCC would be valuable in its early detection, prognostication and development of new strategies for prevention and treatment.

There is accumulating evidence to suggest that the TGF-β signal transduction pathway is involved in head and neck carcinogenesis (Lu et al., Cancer Res 64:4405-10, 2004; Qiu et al., Cancer Lett 245:163-70, 2007). TGF-β is a multifunctional cytokine with diverse biological effects on cellular processes, including cell proliferation, migration, differentiation, and apoptosis. The three mammalian TGF-β isoforms, TGF-β₁, -β₂ and -β₃, exert their functions through a cell surface receptor complex composed of type I (TGFBR1) and type II (TGFBR2) serine/threonine kinase receptors. Receptor activation induces both SMAD proteins and other downstream targets, including Ras, RhoA, TAK1, MEKK1, PI3K, and PP2A, to produce the full spectrum of TGF-β responses (Roberts and Wakefield, Proc Natl Acad Sci USA 100:8621-8623, 2003; Derynck and Zhang, Nature 425:577-584, 2003; Massagué, Cell 134:215-230, 2008). The effects of TGF-β signaling in carcinogenesis largely depend on the tissue of origin and the tumor type. In most types of human cancer, TGF-β plays a paradoxical role in cancer development by acting as a tumor suppressor in early stages (Engle et al., Cancer Res 59:3379-3386, 1999). However, as cells progress towards fully malignant tumor cells, they undergo changes that result in reduced expression of TGF-β receptors, increased expression of TGF-β ligands, and resistance to growth inhibition by TGF-13. Thus, in later stages, TGF-β evokes tumorigenicity and finally promotes tumor metastasis (Pick and Roberts, Adv Cancer Res 83:1-54, 2001; Tang et al., J Clin Invest 112:1116-1124, 2003). Thus, a need remains to further delineate the role of the TGF-β pathway in various types of cancer to aid in the diagnosis, prognosis and treatment of particular cancers, such as head and neck cancer.

### SUMMARY

Disclosed herein is the finding that the PI3K/Akt and TGF-β pathways act cooperatively to promote the development of cancer, particularly squamous cell carcinoma (SCC), such as head and neck squamous cell carcinoma (HNSCC).

Accordingly, provided herein is a method of diagnosing a subject as having cancer, such as squamous cell carcinoma. In some embodiments, the method includes detecting expression of transforming growth factor-β receptor type 1 (TGFBR1) and phosphatase and tensin homolog (PTEN) in a sample obtained from the subject; or detecting the presence or absence of at least one tumor-associated mutation in the TGFBR1 gene and at least one tumor-associated mutation in the PTEN gene. A decrease in expression of TGFBR1 and PTEN in the sample; or the presence of the at least one mutation in TGFBR1 and the at least one mutation in PTEN in the sample, indicates the subject has SCC, or has increased susceptibility to developing SCC. In some embodiments of the diagnostic method, the subject diagnosed with SCC is treated for SCC.

Also provided herein is a method of treating a subject with cancer, such as SCC, by selecting a subject in need of treatment and treating the subject for SCC, for example by administering to the subject a therapeutically effective amount of an inhibitor of the PI3K/Akt pathway and a therapeutically effective amount of a modulator of the TGF-β pathway, wherein administration of the inhibitor and modulator results in reduction in tumor size, inhibition of tumor growth, inhibition of tumor metastasis and/or inhibition of tumor progression, thereby treating the subject diagnosed with cancer.

Pharmaceutical compositions that include an inhibitor of the PI3K/Akt pathway and a modulator of the TGF-β pathway, and the use of such pharmaceutical compositions for the treatment of cancer, such as SCC, are also provided herein.

Further provided are genetically modified mice with a homozygous deletion of the TGFBR1 gene and a homozygous deletion of the PTEN gene. The genetically modified mice are highly susceptible to developing SCC tumors, such as HNSCC tumors. Use of the disclosed genetically modified mice for identifying therapeutic agents for the treatment of SCC is also provided.

The foregoing and other objects, features, and advantages of the invention will become more apparent from the following detailed description, which proceeds with reference to the accompanying figures.

### BRIEF DESCRIPTION OF THE FIGURES

FIG. 1A is a bar graph showing decreased TGF-β signaling in head and neck epithelia of Tgfbr1 cKO mice. Decreased expression of Tgfbr1 mRNA in the bucal mucosa and squamous cell carcinomas (SCCs) of Tgfbr1 cKO mice was determined by qRT-PCR (**, P<0.01 and ***, P<0.001, significantly different from littermate controls). FIG. 1B is a series of digital images of immunostaining of Tgfbr1 and p-Smad2 in the tongue of Tgfbr1^{f/f} and Tgfbr1 cKO mice. Tgfbr1 and p-Smad2 were reduced in the tongue and SCC of Tgfbr1 cKO mice. The dotted lines delineate the adjacent epithelial compartment (Bar, 50 μm). FIG. 1C is a digital image of a Western blot for detection of TGF-β signaling in buccal mucosa and tongue of Tgfbr1^{f/f} and Tgfbr1 cKO mice. Tgfbr1 and p-Smad2 were reduced in buccal mucosa, tongue and SCCs of Tgfbr1 cKO mice compared to that in buccal mucosa and tongue of Tgfbr1^{f/f} mice.

FIGS. 2A-2G are photographs and digital images showing 7,12-dimethylbenz(a)anthracene (DMBA)-initiated Tgfbr1 cKO mice develop HNSCCs. Tumors developed at the oral cavity (A) of Tgfbr1 cKO mice. Shown are pathological sections of oral squamous cell carcinoma (B); infiltrating squamous cell carcinoma (D); and low magnification of the heart (thick black arrow) and lung block (F). Examples of intrapulmonary metastases are indicated by black arrows; extrapulmonary (lymph nodes) with a white arrow; and non-compromised lung parenchyma with a block white arrow. The inset images (C, E and G) depict fine details of the malignant cells. The metastasis (black block arrow) is surrounding by compressed lung parenchyma (white block arrow). The arrow indicates a bronchus. Magnifications are 20× and 200× for main figure and inset, respectively. FIG. 2H is a bar graph showing DMBA-initiated Tgfbr1 cKO mice develop SCCs more frequently compared with Tgfbr1^{f/f} mice. Forty-five percent of Tgfbr1 cKO mice developed SCCs, while no tumors were observed in the heterozygous (K14CreER;Tgfbr1^{f/+}) or the Tgfbr1 flox homozygous (Tgfbr1^{f/f}) control littermates during 1 year of observation after DMBA initiation. 9.7% of Tgfbr1 cKO mice developed spontaneous tumors in the head and neck epithelia without DMBA treatment.

FIG. 3A is a series of digital images of histological sections showing increased expression of Ki67 and loss of apoptosis in the basal layer of tongue of the Tgfbr1 cKO mice 4 weeks after tamoxifen (TM) and DMBA treatment. The dotted lines delineate the adjacent epithelial compartment. Bar, 50 μm. FIG. 3B is a series of digital images of histological sections showing a significantly increased number of proliferative cells in tongue and SCCs of Tgfbr1 cKO mice by BrdU assays. CKDKN1A expression was reduced in tongue and SCCs of Tgfbr1 cKO mice compared to that in Tgfbr1^{f/f} mice. In contrast, c-Myc was overexpressed in tongue of Tgfbr1 cKO mice and its expression was even more remarkable in SCCs. The dotted lines delineate the adjacent epithelial compartment. Bar, 50 μm. FIG. 3C is a Western blot confirming the results shown in FIG. 3B. FIG. 3D is a series of bar graphs showing the percentage of positive cells in tongue or SCCs of Tgfbr1 cKO mice compared with that of Tgfbr1^{f/f} mice (average of three to five immunostained sections; **, P<0.01; ***, P<0.001).

FIG. 4A is a series of digital images of histological sections showing enhanced paracrine effects of TGF-β in Tgfbr1 cKO mice. Significantly increased expression of Cox-2 in SCCs as well as overexpression of Endoglin (CD105), α-SMA in the stroma surrounding SCCs of Tgfbr1 cKO mice (magnifications, 200×). No expression was detected in normal tongue of Tgfbr1^{f/f} or Tgfbr1 cKO mice. The dotted lines delineate the adjacent epithelial compartment. Bar, 50 μm. FIG. 4B is a pair of bar graphs showing the percentage of Cox-2 positive cells and intratumoral microvessel density (iMVD) indicated by Endoglin (CD105)-stained microvessels per 200× field in tumor stroma of Tgfbr1 cKO (five immunostained sections; **, P<0.01; ***, P<0.001). FIG. 4C is a series of digital images of histological sections showing Tgfb1 expression in the tumor stroma by immunofluorescent staining (magnifications, 200×). FIG. 4D is a bar graph showing Tgfb1 mRNA expression by qRT-PCR.

FIG. 5A is a series of digital images showing activation of the PI3K/Akt pathway in SCCs that developed in Tgfbr1 cKO mice. Immunostaining revealed a significantly increased number of positive cells of p-Akt, p-mTOR in the SCCs that developed in Tgfbr1 cKO mice. The dotted lines delineate the adjacent epithelial compartment. Bar, 50 μm. FIG. 5B is a series of Western blots showing that a significantly increased level of unphosphorylated PTEN, an active form of the protein, was detected in SCC that developed in the DMBA-treated Tgfbr1 cKO mice. However, comparable elevated levels of the phosphorylated form of Akt (p-Akt) were also observed in SCC examined by Western blot analysis.

FIG. 6 is a schematic representation of the proposed TGF-β signaling alteration that promotes HNSCC in mice. In normal cells, TGF-β inhibits cell proliferation through Smad-dependent pathway. It also induces apoptosis through repressing the PI3K/Akt pathway resulting in tumor suppression. Decreased Tgfbr1 expression in Tgfbr1 cKO mice leads to increased cell proliferation and cell survival through PTEN independent activation of PI3K/Akt pathway. DMBA treatment which causes H-ras mutation as well as other mechanisms may also play an important role in Akt activation. Decreased TGFBR1 can also increase TGF-β1 in tumor stroma, by as yet unidentified mechanisms, which leads to increased invasion, angiogenesis, inflammation as well as immune suppression through paracrine effect of TGF-β. In summary, inactivation of TGF-β signaling, in the context of ras mutations and aberrant activation of the PI3K/Akt pathway, accompanied by increased paracrine effect of TGF-β, switches TGF-β signaling from tumor suppression in normal cells to tumor promotion in head and neck carcinogenesis of Tgfbr1 cKO mice.

FIG. 7A is a schematic diagram of PCR analysis of Tgfbr1 recombination for conditional deletion of Tgfbr1 in head and neck epithelia after tamoxifen (TM) treatment. The Tgfbr1 genomic locus was targeted for recombination. Black arrows indicate positions of PCR primers. Black arrowheads indicated LoxP sites. FIG. 7B is a digital image of an electrophoretic gel showing specific Tgfbr1 deletion in head and neck epithelia in Tgfbr1 cKO mice. Genomic DNA was extracted from major tissues 10 days after TM treatment. Tgfbr1 deletion was detected in buccal mucosa (BM) and tongue (Tg) as well as ear (Er) of the Tgfbr1 cKO mice. BM=buccal mucosa; Tg=tongue; Es=esophagus; FS=forestomach; Er=ear; SK=back skin; IT=intestine; Lv=liver; Lg=lung; Ht=heart; Br=brain; SG=salivary gland; Sp=spleen; and Kd=kidney.

FIG. 8A is a series of FACS plots showing reduction of effector T cells and immune suppression in Tgfbr1 cKO mice. Compared with their control littermates, Tgfbr1 cKO mice showed significantly reduced amounts of both CD4⁺ and CD8⁺ effector T cells at the same time that the regulatory CD4⁺CD25⁺Foxp3⁺ T cells were increased, indicating the existence of immune suppression in Tgfbr1 cKO mice. FIG. 8B is a digital image of an hematoxylin and eosin (H&E) stain of infiltrative border of a squamous cell carcinoma indicating a chronic inflammatory infiltrate. The inset depicts the mixed nature of the inflammation (magnifications, 20× and 200× for main figure and inset, respectively).

FIG. 9 is a bar graph showing tumor incidence in Tgfbr1/PTEN conditional double knockout mice (Tgfbr1/PTEN COKO), Pten COKO mice, Tgfbr1 COKO mice, and control mice following treatment with tamoxifen (TM).

FIG. 10 is a bar graph showing the percentage of Tgfbr1/PTEN cOKO mice with SCCs in specific sites.

FIGS. 11A and 11B are bar graphs showing the expression level of TGFBR1 mRNA (A) and PTEN mRNA (B) in seven different human HNSCC cell lines, relative to expression in control human oral keratinocyte (HOK) cells.

FIG. 12A is a set of representative immunostains of human HNSCC samples and normal mucosa controls. Tissue array analysis was performed by immunostaining 60 HNSCC samples and 12 normal controls. HNSCC and control tissue samples were immunostained for TGFBR1, phosphorylated Smad (p-Smad2^{S465/467}), PTEN and phosphorylated Akt (p-Akt^{S473}). FIG. 12B is table showing the number of samples exhibiting an increase or decrease in protein expression. TGFBR1 and PTEN protein levels were found to be decreased in 29/60 (48%) and 48/60 (80%) HNSCC samples, respectively. A similar decrease was also observed in phosphorylated Smad2, an activated mediator of TGF-β signaling ( 27/60, 45%) as well as increase in p-Akt, a downstream target inhibited by PTEN ( 35/60, 58%). In total, 26 out of 60 HNSCC samples (43%) exhibited concurrent TGFBR1 and PTEN loss.

FIG. 13A is a gel showing expression of IL-13Rα2 in primary cells taken from TGFBR1 and PTEN cKO tumors of the ear, neck, nose and lip. Primary cells from two tumors are shown (R1PCOKO-1 and R1PCOKO-2). FIGS. 13B and 13C are line graphs showing protein synthesis in R1PCOKO-1 and R1PCOKO-2 derived cells following treatment with various concentrations of cytotoxin. Human HNSCC cells are shown for comparison (PM-RCC).

### DETAILED DESCRIPTION

### I. Abbreviations

BrdU Bromodeoxyuridine
BSA Bovine serum albumin
cKO Conditional knockout
DAPI 4′,6′-diamidino-2-phenylidole
DMBA 7,12-Dimethylbenz[a]anthracene
DNA Deoxyribonucleic acid
ER Estrogen receptor
HNSCC Head and neck squamous cell carcinoma
IHC Immunohistochemistry
iMVD Intratumoral microvessel density
i.p. Intraperitoneal
miRNA MicroRNA
mRNA Messenger RNA
mTOR Mammalian target of rapamycin
PBS Phosphate buffered saline
PCR Polymerase chain reaction
PI3K Phosphoinositide 3-kinase
PP2A Protein phosphatase 2A
PTEN Phosphatase and tensin homolog
PVDF Polyvinylidene fluoride
qRT-PCR Quantitative real time polymerase chain reaction
RNA Ribonucleic acid
SCC Squamous cell carcinoma
shRNA Short hairpin RNA
siRNA Small interfering RNA
TAK1 TGF-β activated kinase 1
TGF-β Transforming growth factor-beta
TGFBR1 Transforming growth factor beta receptor I
TM Tamoxifen
TUNEL Terminal deoxyribonucleotidyl transferase-mediated dUTP nick end labeling

### II. Terms and Methods

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Administration: The introduction of a composition into a subject by a chosen route. For example, if the chosen route is intravenous, the composition is administered by introducing the composition into a vein of the subject. In some embodiments, the route of administration of a pharmaceutical composition is oral, topical or systemic.

AKT: As used herein, the term “AKT” includes AKT1, AKT2 and AKT3. The AKT1 gene encodes a serine-threonine protein kinase that is catalytically inactive in serum-starved primary and immortalized fibroblasts. AKT1 and the related AKT2 are activated by platelet-derived growth factor. The activation, which occurs through phosphatidylinositol 3-kinase, is rapid and specific, and it is abrogated by mutations in the pleckstrin homology domain of AKT1. AKT1 is also known as v-akt murine thymoma viral oncogene homolog 1, PKB; RAC; PRKBA; MGC99656; PKB-ALPHA; and RAC-ALPHA. Nucleotide and amino acid sequences for human AKT1, and AKT1 from other species, are publically available. For example, GenBank Accession Nos. NM_{—}005163.2 and NP_{—}005154.2 are nucleotide and amino acid sequences, respectively, of human AKT1 variant 1; GenBank Accession Nos. NM_{—}001014432.1 and NP_{—}001014432.1 are nucleotide and amino acid sequences, respectively, of human AKT1 variant 2; and GenBank Accession Nos. NM_{—}001014431.1 and NP_{—}001014431.1 are nucleotide and amino acid sequences, respectively, of human AKT1 variant 3.

The AKT2 gene is a putative oncogene encoding a protein belonging to a subfamily of serine/threonine kinases containing SH2-like (Src homology 2-like) domains. The Akt2 protein is a general protein kinase capable of phosphorylating several known proteins. AKT2 is also known as v-akt murine thymoma viral oncogene homolog 2; PKBB; PRKBB; PKBBETA; and RAC-BETA. Nucleotide and amino acid sequences for human AKT2, and AKT2 from other species, are publically available. For example, GenBank Accession Nos. NM_{—}001626.3 and NP_{—}001617.1 are nucleotide and amino acid sequences, respectively, of human AKT2.

AKT3 is a member of the AKT (also called PKB) serine/threonine protein kinase family. AKT kinases are known to be regulators of cell signaling in response to insulin and growth factors. They are involved in a wide variety of biological processes including cell proliferation, differentiation, apoptosis, tumorigenesis, as well as glycogen synthesis and glucose uptake. The Akt3 protein kinase has been shown to be stimulated by platelet-derived growth factor (PDGF), insulin, and insulin-like growth factor 1 (IGF1). AKT3 is also known as v-akt murine thymoma viral oncogene homolog 3; protein kinase B, gamma; PKBG; PRKBG; STK-2; PKB-GAMMA; RAC-gamma; RAC-PK-gamma; and DKFZp434N0250. Nucleotide and amino acid sequences for human AKT3, and AKT3 from other species, are publically available. For example, GenBank Accession Nos. NM_{—}005465.3 and NP_{—}005456.1 are nucleotide and amino acid sequences, respectively, of human AKT3 isoform 1; and GenBank Accession Nos. NM_{—}181690.1 and NP_{—}859029.1 are nucleotide and amino acid sequences, respectively, of human AKT3 isoform 2.

Each of the GenBank Accession numbers listed above is incorporated by reference as it appears in the GenBank database on Apr. 24, 2009.

Analog: A molecule that is structurally and functionally related to another molecule.

Antibody: A polypeptide ligand comprising at least a light chain or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody.

Antibodies include intact immunoglobulins and the variants and portions of antibodies well known in the art, such as Fab fragments, Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W. H. Freeman & Co., New York, 1997.

Typically, a naturally occurring immunoglobulin has heavy (H) chains and light (L) chains interconnected by disulfide bonds. There are two types of light chain, lambda (λ) and kappa (k). There are five main heavy chain classes (or isotypes) which determine the functional activity of an antibody molecule: IgM, IgD, IgG, IgA and IgE.

Each heavy and light chain contains a constant region and a variable region (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs.” The extent of the framework region and CDRs have been defined (see, Kabat et al., Sequences of Proteins of Immunological Interest, U.S. Department of Health and Human Services, 1991). The Kabat database is now maintained online. The sequences of the framework regions of different light or heavy chains are relatively conserved within a species, such as humans. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “chimeric antibody” has framework residues from one species, such as human, and CDRs (which generally confer antigen binding) from another species, such as a murine antibody.

A “humanized” immunoglobulin is an immunoglobulin including a human framework region and one or more CDRs from a non-human (for example a mouse, rat, or synthetic) immunoglobulin. The non-human immunoglobulin providing the CDRs is termed a “donor,” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by means of genetic engineering (see for example, U.S. Pat. No. 5,585,089).

A “human” antibody (also called a “fully human” antibody) is an antibody that includes human framework regions and all of the CDRs from a human immunoglobulin. In one example, the framework and the CDRs are from the same originating human heavy and/or light chain amino acid sequence. However, frameworks from one human antibody can be engineered to include CDRs from a different human antibody. All parts of a human immunoglobulin are substantially identical to corresponding parts of natural human immunoglobulin sequences.

Antisense compound: Refers to an oligomeric compound that is at least partially complementary to the region of a target nucleic acid molecule to which it hybridizes. As used herein, an antisense compound that is “specific for” a target nucleic acid molecule is one which specifically hybridizes with and modulates expression of the target nucleic acid molecule. As used herein, a “target” nucleic acid is a nucleic acid molecule to which an antisense compound is designed to specifically hybridize and modulate expression.

Nonlimiting examples of antisense compounds include primers, probes, antisense oligonucleotides, siRNAs, miRNAs, shRNAs and ribozymes. As such, these compounds can be introduced as single-stranded, double-stranded, circular, branched or hairpin compounds and can contain structural elements such as internal or terminal bulges or loops. Double-stranded antisense compounds can be two strands hybridized to form double-stranded compounds or a single strand with sufficient self complementarity to allow for hybridization and formation of a fully or partially double-stranded compound. In particular examples herein, the antisense compound is an antisense oligonucleotide, siRNA, miRNA, shRNA or ribozyme.

Antisense oligonucleotide: As used herein, an “antisense oligonucleotide” is a single-stranded antisense compound that is a nucleic acid-based oligomer. An antisense oligonucleotide can include one or more chemical modifications to the sugar, base, and/or internucleoside linkages. Generally, antisense oligonucleotides are “DNA-like” such that when the antisense oligonucleotide hybridizes to a target RNA molecule, the duplex is recognized by RNase H (an enzyme that recognizes DNA:RNA duplexes), resulting in cleavage of the RNA.

Chemotherapeutic agents: Any chemical agent with therapeutic usefulness in the treatment of diseases characterized by abnormal cell growth. Such diseases include tumors, neoplasms, and cancer as well as diseases characterized by hyperplastic growth such as psoriasis. In some embodiments, a chemotherapeutic agent is an agent of use in treating a squamous cell carcinoma, such as head and neck squamous cell carcinoma. In some embodiments, a chemotherapeutic agent is a radioactive compound. One of skill in the art can readily identify a chemotherapeutic agent of use (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer, L., Berkery, R. (eds.): Oncology Pocket Guide to Chemotherapy, 2nd ed. St. Louis, Mosby-Year Book, 1995; Fischer, D. S., Knobf, M. F., Durivage, H. J. (eds): The Cancer Chemotherapy Handbook, 4th ed. St. Louis, Mosby-Year Book, 1993). Combination chemotherapy is the administration of more than one agent to treat cancer. One example is the administration of an inhibitor of the PI3K/Akt pathway and a modulator of the TGF-β pathway used in combination with a radioactive or chemical compound.

Control: A “control” refers to a sample or standard used for comparison with an experimental sample, such as a sample obtained from a subject to be tested for HNSCC. In some embodiments, the control is a sample obtained from a healthy patient. In some embodiments, the control is a historical control or reference standard (i.e. a previously tested control sample or group of samples that represent baseline or normal values, such as the level of TGFBR1 or PTEN expression in non-tumor tissue). In some cases, a control can also refer to a wild-type gene or protein (or sample containing a wild-type gene or protein), such as a wild-type TGFBR1 or PTEN.

Derivative: A chemical compound derived from another compound either directly or by modification or partial substitution.

Detecting expression of a gene product: Determining the existence, in either a qualitative or quantitative manner, of a particular nucleic acid or protein product. Exemplary methods of detecting expression include microarray analysis, RT-PCR, Northern blot, Western blot, immunohistochemistry, ELISA and mass spectrometry.

Detecting the level of expression: As used herein, “detecting the level” of mRNA or protein expression refers to quantifying the amount of a particular mRNA or protein (such as TGFBR1 or PTEN mRNA or protein) present in a sample. Detecting expression of mRNA or protein can be achieved using any method known in the art or described herein, such as by RT-PCR (for mRNA) or immunoblot (for protein).

Head and neck squamous cell carcinoma (HNSCC): Cancer of the head and neck that begins in squamous cells (thin, flat cells that form the surface of the skin, eyes, various internal organs, and the lining of hollow organs and ducts of some glands). Squamous cell carcinoma of the head and neck includes cancers of the nasal cavity, sinuses, lips, mouth, salivary glands, throat, and larynx (voice box). Most head and neck cancers are squamous cell carcinomas.

Inhibitor: As used herein, the term “inhibitor” includes any type of molecule that inhibits the expression or activity of a target gene or protein. An inhibitor can be any type of compound, such as a small molecule, antibody or antisense compound. In some embodiments, the target gene or protein is a member of the TGF-β or PI3K/Akt pathway.

Inhibitor of TGF-β or TGF-β receptor: Any compound that directly or indirectly inhibits expression or activity of TGF-β or TGF-β receptor. An inhibitor of TGF-β can inhibit all isoforms (TGF-β₁, TGF-β₂, TGF-β₃), or a single isoform. Similarly, an inhibitor of TGF-β receptor can inhibit both type I and type II TGF-β receptors, or a single type of TGF-β receptor. In some embodiments herein, the TGF-β or TGF-β receptor inhibitor is an antibody, such as, but not limited to, CAT-192 (a monoclonal antibody specific for human TGF-β₁); CAT-152 (a monoclonal antibody specific for human TGF-β₂); 1D11 (a monoclonal antibody that inhibits TGF-β₁ and TGF-β₂); or 2G7 (a pan-TGF-β monoclonal antibody). In some embodiments herein, the TGF-β or TGF-β receptor inhibitor is a polypeptide, such as, but not limited to, sTbRII:Fc (soluble transmembrane domain of TGF-β receptor II fused to Fc; binds TGF-β₁ and TGF-β₃); or betaglycan (also known as TGF-β receptor III; binds to various members of the TGF-β family of ligands; is not involved directly in TGF-β signal transduction, but acts as a reservoir for ligands of TGF-β receptors. In some embodiments, the TGF-β or TGF-β receptor inhibitor is a small molecule, such as, but not limited to, SB-431542 (inhibitor of TGF-β receptor II; 4-(5-Benzol[1,3]dioxol-5-yl-4-pyrldin-2-yl-1H-imidazol-2-yl)-benzamide hydrate, 4-[4-(3,4-Methylenedioxyphenyl)-5-(2-pyridyl)-1H-imidazol-2-yl]-benzamide hydrate, 4-[4-(1,3-Benzodioxol-5-yl)-5-(2-pyridinyl)-1H-imidazol-2-yl]-benzamide hydrate); NPC-30345 (inhibitor of TGF-β receptor I; Scios Inc., Fremont, Calif.); LY364947 (inhibitor of TGF-β receptor I; 4-[3-(2-Pyridinyl)-1H-pyrazol-4-yl]-quinoline); A-83-01 (inhibitor of TGF-β receptor type I; 3-(6-Methylpyridin-2-yl)-1-phenylthiocarbamoyl-4-quinolin-4-ylpyrazole); LY550410 (inhibitor of TGF-β receptor type I; Lilly Research); LY580276 (inhibitor of TGF-13 receptor type I; Lilly Research); LY566578 (inhibitor of TGF-β receptor type I; Lilly Research); SB-505124 (selective inhibitor of TGF-β receptor type I; 2-(5-benzo[1,3]dioxol-5-yl-2-tert-butyl-3H-imidazol-4-yl)-6-methylpyridine hydrochloride); SD-093 (inhibitor of TGF-β receptor type I; Scios Inc.); or SD-208 (inhibitor of TGF-β receptor type I; Scios Inc.). In some embodiments, the TGF-β or TGF-β receptor inhibitor is an antisense compound, such as, but not limited to, AP-12009 (antisense oligonucleotide specific for TGF-β₂; Antisense Pharma, Regensburg, Germany) or AP-11014 (antisense oligonucleotide specific for TGF-β₁; Antisense Pharma).

LY294002: A selective small molecule inhibitor of PI3K. LY294002 is also known as 2-(4-morpholinyl)-8-phenyl-4H-1-benzopyran-4-one (Vlahos et al., J Biol Chem 269:5241-5248, 1994). The molecular formula of LY294002 is C₁₉H₁₇NO₃.

Mammalian target of rapamycin (mTOR): A serine/threonine kinase that regulates the expression of proteins involved in cell growth and proliferation via phosphorylation of specific substrates. As such, mTOR plays an integral role in the response to numerous hormones and growth factors. Synonyms for mTOR include FRAP1, FKBP12-rapamycin complex-associated protein, FK506-binding protein 12-rapamycin complex-associated protein 1, rapamycin target protein and RAPT1. Nucleotide and amino acid sequences of mTOR are known in the art (for example, Genbank Accession Nos. NM_{—}004958 and BC117166). Each of the GenBank Accession numbers listed herein is incorporated by reference as it appears in the GenBank database on Apr. 24, 2009.

MicroRNA (miRNA): Single-stranded RNA molecules that regulate gene expression. miRNAs are generally 21-23 nucleotides in length. miRNAs are processed from primary transcripts known as pri-miRNA, to short stem-loop structures called pre-miRNA, and finally to functional miRNA. Mature miRNA molecules are partially complementary to one or more messenger RNA molecules, and their primary function is to down-regulate gene expression. MicroRNAs regulate gene expression through the RNAi pathway.

Modulator of the TGF-β pathway: A compound that either inhibits (e.g., decreases or downregulates) or activates (e.g., increases or upregulates) expression or activity of a member of the TGF-β pathway (such as TGF-β or a TGF-β receptor). In some embodiments, the modulator of the TGF-β pathway is an inhibitor selected from CAT-192, CAT-152, ID11, 2G7, sTbRII:Fc, betaglycan, SB-431542, NPC-30345, LY364947 and AP-12009.

mTOR inhibitor: A molecule that inhibits expression or activity of mTOR. mTOR inhibitors include, but are not limited to small molecule, antibody, peptide and nucleic acid inhibitors. For example, an mTOR inhibitor can be a molecule that inhibits the kinase activity of mTOR or inhibits binding of mTOR to a ligand. Inhibitors of mTOR also include molecules that down-regulate expression of mTOR, such as an antisense compound. A number of mTOR inhibitors are known in the art and are discussed below. In some embodiments, the mTOR inhibitor is rapamycin or a rapamycin analog.

Mutation: Any change of the DNA sequence within a gene or chromosome. In some instances, a mutation will alter a characteristic or trait (phenotype), but this is not always the case. Types of mutations include base substitution point mutations (e.g., transitions or transversions), deletions, and insertions. Missense mutations are those that introduce a different amino acid into the sequence of the encoded protein; nonsense mutations are those that introduce a new stop codon. In the case of insertions or deletions, mutations can be in-frame (not changing the frame of the overall sequence) or frame shift mutations, which may result in the misreading of a large number of codons (and often leads to abnormal termination of the encoded product due to the presence of a stop codon in the alternative frame).

This term specifically encompasses variations that arise through somatic mutation, for instance those that are found only in disease cells, but not constitutionally, in a given individual. Examples of such somatically-acquired variations include the point mutations that frequently result in altered function of various genes that are involved in development of cancers. This term also encompasses DNA alterations that are present constitutionally, that alter the function of the encoded protein in a readily demonstrable manner, and that can be inherited by the children of an affected individual. In this respect, the term overlaps with “polymorphism,” but generally refers to the subset of constitutional alterations that have arisen within the past few generations in a kindred and that are not widely disseminated in a population group.

A “conditional mutation” is a mutation that is present only upon exposure to a particular environmental stimulus, compound or other condition. In some embodiments disclosed herein, a genetically modified mouse has a conditional mutation in both TGFBR1 and PTEN. Exposure of the mouse oral cavity to tamoxifen induces Cre expression, leading to conditional deletion both TGFBR1 and PTEN in the mouse head and neck epithelia.

Pharmaceutical agent or pharmaceutical composition: A compound or composition capable of inducing a desired therapeutic or prophylactic effect when properly administered to a subject or a cell. Pharmaceutical agents can include chemical and/or biological agents.

Pharmaceutically acceptable carriers: The pharmaceutically acceptable carriers of use are conventional. Remington's Pharmaceutical Sciences, by E. W. Martin, Mack Publishing Co., Easton, Pa., 15th Edition, 1975, describes compositions and formulations suitable for pharmaceutical delivery of the compositions disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Phosphoinositide-3 kinase (PI3K): A family of related enzymes that are capable of phosphorylating the 3 position hydroxyl group of the inositol ring of phosphatidylinositol. PI3Ks are also known as phosphatidylinositol-3-kinases. Class I PI3K are heterodimeric molecules composed of a regulatory subunit and a catalytic subunit. Class II and Class III PI3K are differentiated from Class I by their structure and function. Class II PI3K are composed of one of three catalytic isoforms (C2α, C2β, and C2γ), but have no regulatory proteins. Class III PI3K exist as a heterodimers of a catalytic subunit (Vps34) and a regulatory (p150) subunit. Genes encoding PIK3 subunits include, for example, PIK3C2A, PIK3C2B, PIK3C2G, PIK3C3, PIK3CA, PIK3CB, PIK3CG, PIK3CD, PIK3R1, PIK3R2, PIK3R3, PIK3R4, PIK3R4, PIK3R5 and PIK3R6.

Phosphatase and tensin homolog (PTEN): A tumor suppressor that is mutated in a large number of cancers at high frequency. The protein encoded by this gene is a phosphatidylinositol-3,4,5-trisphosphate 3-phosphatase. It contains a tensin like domain as well as a catalytic domain similar to that of the dual specificity protein tyrosine phosphatases. Unlike most of the protein tyrosine phosphatases, this protein preferentially dephosphorylates phosphoinositide substrates. PTEN negatively regulates intracellular levels of phosphatidylinositol-3,4,5-trisphosphate in cells and functions as a tumor suppressor by negatively regulating the AKT/PKB signaling pathway. PTEN is also known as BZS, MHAM, TEP1, MMAC1, PTEN1, 10q23del and MGC11227. Nucleotide and amino acid sequences for human PTEN, and PTEN from other species, are publically available. For example, GenBank Accession Nos. NM_{—}000314.4 and NP_{—}000305.3 are nucleotide and amino acid sequences, respectively, of human PTEN. Each of the GenBank Accession numbers listed above is incorporated by reference as it appears in the GenBank database on Apr. 24, 2009.

PI3K/Akt pathway: A signaling pathway involved in a number of cellular processes, such as cell growth, proliferation, differentiation, motility, survival, intracellular trafficking, metabolism and angiogenesis. In the context of the present disclosure, members of the PI3K/Akt pathway include, but are not limited to, PI3K, Akt, PTEN, PDK1 and mTOR.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Prostate Cancer: A malignant tumor, generally of glandular origin, of the prostate. Prostate cancers include adenocarcinomas and small cell carcinomas. Many prostate cancers express prostate specific antigen (PSA).

Pyruvate dehydrogenase kinase (PDK1): A mitochondrial multienzyme complex that catalyzes the oxidative decarboxylation of pyruvate and is one of the major enzymes responsible for the regulation of homeostasis of carbohydrate fuels in mammals. The enzymatic activity of PDK1 is regulated by a phosphorylation/dephosphorylation cycle. Nucleotide and amino acid sequences for human PDK1, and PDK1 from other species, are publically available. For example, GenBank Accession Nos. NM_{—}002610.3 and NP_{—}002601.1 are nucleotide and amino acid sequences, respectively, of human PDK1. Each of the GenBank Accession numbers listed herein is incorporated by reference as it appears in the GenBank database on Apr. 24, 2009.

Rapamycin: A small molecule with known immunosuppressive and anti-proliferative properties. Rapamycin, also known as sirolimus, is a macrolide that was first discovered as a product of the bacterium Streptomyces hygroscopicus. Rapamycin binds and inhibits the activity of mTOR. The chemical formula of rapamycin is C₅₁H₇₉NO₁₃ and the International Union of Pure and Applied Chemistry (IUPAC) name is (3S,6R,7E,9R,10R,12R,14S,15E,17E,19E,21S,23S,26R,27R,34aS)-9,10,12,13,14,21,22,23,24,25,26,27,32,33,34,34a-hexadecahydro-9,27-dihydroxy-3-[(1R)-2-[(1S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4]-oxaazacyclohentriacontine-1,5,11,28,29(4H,6H,31H)-pentone. Analogs of rapamycin are known and include, for example, CCI-779 (also called temsirolimus and Torisel™) and RAD-001 (also known as 42-O-(2-hydroxy)ethyl rapamycin and everolimus).

Reduced activity: As used herein, “reduced activity” of a mutant protein (such as a mutant TGFBR1 or PTEN protein) refers to a reduction in any normal function or activity of the protein relative to the wild-type version of the protein. For example, reduced activity of a TGFBR1 protein can include, for example, a reduction in the signaling capability of the receptor, a reduction in kinase activity or a reduction in the ability of the receptor to form dimers with TGFBR2. A reduction in PTEN protein activity can include, for example, a reduction in phosphatase activity, a reduction in tumor suppressor activity or a reduction in its ability to regulate downstream targets, such as Akt.

Ribozyme: A catalytic RNA molecule. In some cases, ribozymes can bind to specific sites on other RNA molecules and catalyze the hydrolysis of phosphodiester bonds in the RNA molecules.

RNA interference (RNAi): Refers to a cellular process that inhibits expression of genes, including cellular and viral genes. RNAi is a form of antisense-mediated gene silencing involving the introduction of double stranded RNA-like oligonucleotides leading to the sequence-specific reduction of RNA transcripts. Double-stranded RNA molecules that inhibit gene expression through the RNAi pathway include siRNAs, miRNAs, and shRNAs.

Sample or biological sample: As used herein, a “sample” obtained from a subject refers to a cell, fluid or tissue sample. Bodily fluids include, but are not limited to, blood, serum, urine and saliva. In some examples, the sample is a tissue sample comprising epithelial cells obtained from the head or neck of a subject.

Screening: As used herein, “screening” refers to the process used to evaluate and identify candidate agents that are useful for the treatment of cancer, such as SCC. In some embodiments, a candidate agent useful for treatment of cancer is an agent that inhibits tumor growth, inhibits tumor metastasis, reduces tumor size or inhibits tumor progression.

Short hairpin RNA (shRNA): A sequence of RNA that makes a tight hairpin turn and can be used to silence gene expression via the RNAi pathway. The shRNA hairpin structure is cleaved by the cellular machinery into siRNA.

Small interfering RNA (siRNA): A double-stranded nucleic acid molecule that modulates gene expression through the RNAi pathway. siRNA molecules are generally 20-25 nucleotides in length with 2-nucleotide overhangs on each 3′ end. However, siRNAs can also be blunt ended. Generally, one strand of a siRNA molecule is at least partially complementary to a target nucleic acid, such as a target mRNA. siRNAs are also referred to as “small inhibitory RNAs.”

Small molecule inhibitor: A molecule, typically with a molecular weight less than about 1000 Daltons, or in some embodiments, less than about 500 Daltons, wherein the molecule is capable of inhibiting, to some measurable extent, an activity of a target molecule.

Squamous cell carcinoma (SCC): A type of cancer that begins in squamous cells, which are thin, flat cells that look like fish scales. Squamous cells are found in the tissue that forms the surface of the skin, the lining of the hollow organs of the body, and the passages of the respiratory and digestive tracts. SCC is also called epidermoid carcinoma.

Subject: Living multi-cellular vertebrate organisms, a category that includes both human and veterinary subjects, including human and non-human mammals (including research subjects such as rodents). A subject is also referred to herein as a “patient.”

Susceptible (to a disease): At risk of developing a disease. As used herein, a subject that is “highly susceptible” to a disease, such as SCC, is a subject that is at very high risk of developing the disease. In some embodiments, a subject at high risk of developing a disease is a subject that has a 75% or greater chance of developing the disease. As used herein, a subject that has “increased susceptibility” to a disease is an individual that is more likely to develop the disease because of a particular risk factor (such as the presence of a mutation in TGFBR1 and/or PTEN).

Therapeutic agent: A chemical compound, small molecule, or other composition, such as an antisense compound, antibody, protease inhibitor, hormone, chemokine or cytokine, capable of inducing a desired therapeutic or prophylactic effect when properly administered to a subject. “Incubating” includes a sufficient amount of time for an agent to interact with a cell or tissue. “Contacting” includes incubating an agent in solid or in liquid form with a cell or tissue. “Treating” a cell or tissue with an agent includes contacting or incubating the agent with the cell or tissue.

Therapeutically effective amount: A quantity of a specific substance sufficient to achieve a desired effect in a subject being treated. For an example not intended to limit the scope of the disclosure, this can be the amount of a pharmaceutical composition comprising an inhibitor of the PI3K/Akt pathway and a modulator of the TGF-b pathway to reduce tumor size, inhibit tumor growth, inhibit tumor metastasis and/or inhibit tumor progression.

Transforming growth factor-β (TGF-β): A secreted, multi-functional protein that regulates proliferation, cellular differentiation and a number of other cellular functions. Many cells synthesize TGF-β and nearly all cells express receptors for TGF-13. The term “TGF-β” refers to three different protein isoforms, TGF-β₁, TGF-β₂ and TGF-β₃, encoded by the genes TGFB1, TGFB2, TGFB3, respectively. Nucleotide and amino acid sequences for human TGFB, and TGFB from other species, are publically available. For example, GenBank Accession Nos. NM_{—}000660.3 and NP_{—}000651.3 are nucleotide and amino acid sequences, respectively, of human TGFB1; GenBank Accession Nos. NM_{—}001135599.1 and NP_{—}001129071.1 are nucleotide and amino acid sequences, respectively, of human TGFB2 isoform 1; GenBank Accession Nos. NM_{—}003238.2 and NP_{—}003229.1 are nucleotide and amino acid sequences, respectively, of human TGFB2 isoform 2; and GenBank Accession Nos. NM_{—}003239.2 and NP_{—}003230.1 are nucleotide and amino acid sequences, respectively, of human TGFB3. Each of the GenBank Accession numbers listed above is incorporated by reference as it appears in the GenBank database on Apr. 24, 2009.

TGF-β pathway: A signaling pathway involved in a number of cellular processes, such as cell proliferation, differentiation and apoptosis. Members of the TGF-β pathway include, but are not limited to TGF-β₁, TGF-β₂, TGF-β₃ and TGF-β receptor type I and TGF-β receptor type II.

TGF-β receptor: The term “TGF-β receptor” includes TGF-β receptor type I (encoded by TGFBR1) and TGF-β receptor type II (encoded by TGFBR2). TGF-β receptors are serine/threonine protein kinases. The type I and type II TGF-β receptors form a heterodimeric complex when bound to TGF-β, transducing the TGF-β signal from the cell surface to the cytoplasm. TGFBR1 is also known as AAT5; ALK5; SKR4; ALK-5; LDS1A; LDS2A; TGFR-1; ACVRLK4; and transforming growth factor beta, receptor 1. Nucleotide and amino acid sequences for human TGFBR1, and TGFBR1 from other species, are publically available. For example, GenBank Accession Nos. NM_{—}004612.2 and NP_{—}004603.1 are nucleotide and amino acid sequences, respectively, of human TGFBR1, isoform 1; and GenBank Accession Nos. NM_{—}001130916.1 and NP_{—}001124388.1 are nucleotide and amino acid sequences, respectively, of human TGFBR1, isoform 2. TGFBR2 is also known as AAT3; FAA3; MFS2; RIIC; LDS1B; LDS2B; TAAD2; TGFR-2; TGFbeta-RII; transforming growth factor beta, receptor 2. Nucleotide and amino acid sequences for human TGFBR2, and TGFBR2 from other species, are publically available. For example, GenBank Accession Nos. NM_{—}001024847.2 and NP_{—}001020018.1 are nucleotide and amino acid sequences, respectively, of human TGFBR2, isoform A; and GenBank Accession Nos. NM_{—}003242.5 and NP_{—}003233.4 are nucleotide and amino acid sequences, respectively, of human TGFBR2, isoform B. Each of the GenBank Accession numbers listed herein is incorporated by reference as it appears in the GenBank database on Apr. 24, 2009.

Transgenic animal: A non-human animal, usually a mammal, having a non-endogenous (heterologous) nucleic acid sequence present as an extrachromosomal element in a portion of its cells or stably integrated into its germ line DNA (i.e., in the genomic sequence of most or all of its cells). Heterologous nucleic acid is introduced into the germ line of such transgenic animals by genetic manipulation of, for example, embryos or embryonic stem cells of the host animal according to methods well known in the art. A “transgene” is meant to refer to such heterologous nucleic acid, such as, heterologous nucleic acid in the form of an expression construct (such as for the production of a “knock-in” transgenic animal) or a heterologous nucleic acid that upon insertion within or adjacent to a target gene results in a decrease in target gene expression (such as for production of a “knock-out” transgenic animal). A “knock-out” of a gene means an alteration in the sequence of the gene that results in a decrease of function of the target gene, preferably such that target gene expression is undetectable or insignificant. Transgenic knock-out animals can comprise a heterozygous knock-out of a target gene, or a homozygous knock-out of a target gene. “Knock-outs” also include conditional knock-outs, where alteration of the target gene can occur upon, for example, exposure of the animal to a substance that promotes target gene alteration, introduction of an enzyme that promotes recombination at the target gene site (for example, Cre in the Cre-lox system), or other method for directing the target gene alteration postnatally. Transgenic animals are also referred to herein as “genetically modified” animals.

Tumor: All neoplastic cell growth and proliferation, whether malignant or benign, and all pre-cancerous and cancerous cells and tissues. In a non-limiting example, a tumor is a SCC tumor, such as a HNSCC tumor.

Tumor-associated mutation: Any mutation in a gene or protein that is linked to the development, progression or severity of a tumor, such as a HNSCC tumor. In some examples, of the methods disclosed herein, the tumor-associated mutation in TGFBR1 is TGFBR1 (6A). “TGFBR1 (6A)” refers to an in-frame deletion of three alanine residues within a 9-alanine repeat at the 3′-end of the exon 1 coding sequence (Pasche et al., Cancer Res. 58:2727-2732, 1998; Pasche et al., JAMA 294(13):1634-1646, 2005). In other examples, the tumor-associated mutation in TGFBR1 is TGFBR1 (10A). “TGFBR1 (10A)” refers to an in-frame insertion of one alanine residue in the extracellular domain (Pasche et al., Cancer Res. 58:2727-2732, 1998). In some examples, the tumor-associate mutation in PTEN is a missense mutation in exon 5, 6, 7 or 8 (Poetsch et al., Cancer Genet. Cytogenet. 132(1):20-24, 2002).

UCN-01 (7-hydroxystaurosporine): A synthetic derivative of staurosporine with antineoplastic activity. UCN-01 inhibits many phosphokinases, including AKT, calcium-dependent protein kinase C, and cyclin-dependent kinases. The chemical structure name of UCN-01 is 8,12-epoxy-1H,8H-2,7b,12a-triazadibenzo[a, g]cyclonona[cde]trinden-1-one, 2,3,9,10,11,12-hexahydro-3-hydroxy-9-methoxy-8-methyl-10-(methylamino).

Wortmannin: A furanosteroid metabolite of the fungi Penicillium funiculosum, Talaromyces (Penicillium) wortmannii, is a specific, covalent inhibitor of PI3K. The molecular formula of wortmannin is C₂₃H₂₄O₈.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All GenBank Accession Nos. mentioned herein are incorporated by reference in their entirety as they appear in the database as of Apr. 24, 2009. In case of conflict, the present specification, including explanations of terms, will control. In addition, the materials, methods, and examples are illustrative only and not intended to be limiting.

### III. Overview of Several Embodiments

Disclosed herein is the finding that the PI3K/Akt and TGF-β pathways act cooperatively to promote development of cancer, particularly SCC, such as HNSCC. In particular, described is the finding that conditional deletion of TGFBR1 and PTEN in head and neck epithelia of mice leads to spontaneous development of SCC in the mice with complete penetrance. It is demonstrated that mice with conditional deletions of TGFBR1 and PTEN develop tumors in a variety of locations, including, but not limited to the ears, muzzle, oral cavity, tongue, skin, perianal region, penis/vagina, prostate, peri-anal region and periorbital region. In several embodiments, the cancer that develops is a SCC. In some embodiments, the cancer is another type of cancer, such as an adenocarcinoma (for example, prostate cancer).

For many types of cancer, TGF-β plays a paradoxical role by acting as a tumor suppressor at early stages of disease and as a tumor promoter in later stages of cancer. While not wishing to be bound by any particular theory, it is believed that TGF-β acts as an early tumor suppressor, but in the absence of TGFBR1, elevated levels of TGF-β in the tumor stroma create a microenvironment for tumor promotion.

Thus, provided herein is a method of diagnosing a subject as having cancer, such as SCC, or being susceptible to developing cancer, by detecting expression of TGFBR1 and PTEN in a sample obtained from the subject. A decrease in expression of TGFBR1 and PTEN in the sample indicates the subject has cancer, or has increased susceptibility to developing cancer. Also provided herein is a method of diagnosing a subject as having cancer, such as SCC, or being susceptible to developing cancer, by detecting the presence or absence of at least one tumor-associated mutation in the TGFBR1 gene and at least one tumor-associated mutation in the PTEN gene. The presence of the at least one mutation in TGFBR1 and the at least one mutation in PTEN indicates the subject has cancer, or has increased susceptibility to developing cancer. In some embodiments, the cancer is a SCC. In some examples, the SCC is a HNSCC. In some examples, the SCC is a SCC of the skin, oral mucosa, tongue, peri-orbital region, penis, vagina, cervix or peri-anal region. In some embodiments disclosed herein, the cancer is an adenocarcinoma, such as prostate cancer.

In some embodiments, provided herein is a method of diagnosing a subject as having SCC, or being susceptible to developing SCC, by detecting the presence or absence of at least one tumor-associated mutation in the TGFBR1 gene and at least one tumor-associated mutation in the PTEN gene. The presence of the at least one mutation in TGFBR1 and the at least one mutation in PTEN in the sample, indicates the subject has SCC, or has increased susceptibility to developing SCC.

In some embodiments, the at least one tumor-associated mutation in the TGFBR1 gene results in a decrease in expression of TGFBR1 mRNA or results in expression of a TGFBR1 protein with reduced activity; and the at least one tumor-associated mutation in the PTEN gene results in a decrease in expression of PTEN mRNA or results in expression of a PTEN protein with reduced activity.

In some embodiments of the methods, the tumor-associated mutation in the TGFBR1 gene is a complete or partial deletion of TGFBR1. In some examples, the tumor-associated mutation in the TGFBR1 gene is TGFBR1 (6A), which is an in-frame deletion of three alanine residues within a 9-alanine repeat at the 3′-end of the exon 1 coding sequence. In other embodiments, the tumor-associated mutation in the TGFBR1 gene is TGFBR1 (10A), which is an in-frame insertion of one alanine residue in the extracellular domain of TGFBR1. In some embodiments, the tumor-associated mutation in the PTEN gene is a complete or partial deletion of PTEN. In some embodiments, the tumor-associated mutation in the PTEN gene is a missense mutation in exon 5, 6, 7 or 8. Although several exemplary TGFBR1 and PTEN mutations are provided herein, the tumor-associated mutation can be any type of mutation (such as an insertion, deletion or substitution) in a TGFBR1 or PTEN gene or protein that is linked to the development, progression or severity of cancer, such as SCC.

Methods of detecting mutations in a gene or protein are well known in the art and are described in further detail below. Exemplary methods of detecting a mutation in a gene include, but are not limited to, DNA sequencing, oligonucleotide hybridization, PCR, RT-PCR, in situ hybridization, Southern blot, Northern blot, microarray analysis, or other DNA/RNA hybridization platforms. Exemplary methods of detecting a mutation in a protein include, for example, immunoassays (such as ELISA, Western blot or immunoprecipitation) or biochemical assays.

In some embodiments, provided herein is a method of diagnosing a subject as having SCC, or being susceptible to developing SCC, by detecting expression of TGFBR1 and PTEN in a sample obtained from the subject. A decrease in expression of TGFBR1 and PTEN relative to a control indicates the subject has SCC, or has increased susceptibility to developing SCC.

In some embodiments, detecting expression of TGFBR1 and PTEN in a sample comprises detecting the level of TGFBR1 and PTEN mRNA in the sample.

In other embodiments, detecting expression of TGFBR1 and PTEN in a sample comprises detecting the level of TGFBR1 and PTEN protein in the sample. The control can be a suitable control for comparison of mRNA or protein expression. In some examples, the control is a sample obtained from a healthy control subject. In other examples, the control is a reference standard.

Methods of detecting expression of a gene, such as detecting expression of mRNA or protein, are well known in the art and are described in further detail below. Exemplary methods of detecting expression of mRNA include RT-PCR, Northern blotting, RNAse protection assays, or in situ hybridization. Exemplary methods of detecting expression of a protein include ELISA, Western blotting or immunoprecipitation.

In some embodiments of the methods, the sample is a fluid sample, such as a blood sample. In other embodiments, the sample is a cell or tissue sample, such as a sample of epithelia cells obtained from the head or neck region of the subject.

In some embodiments of the methods for diagnosing cancer, such as SCC, in a subject, the method further comprises displaying the diagnostic results using an output device. In some examples, the output device is a computer screen. In other examples, the output device is a printer. In some embodiments, the method further comprises recording the diagnostic results in the subject's electronic medical record.

In some embodiments, if the diagnostic test indicates the subject has SCC, or is susceptible to developing SCC, the subject is subjected to additional diagnostic tests to confirm the diagnosis by other means. Alternatively, the test is used to confirm a diagnosis already indicated by other means. The other means can include diagnostic modalities such as physical examination, clinical suspicion, analysis of additional mutations associated with SCC or a specific sub-type of SCC (such as HNSCC), or histological examination, for example tissue biopsy with histological diagnosis by a pathologist.

In some embodiments of the methods for diagnosing cancer, such as SCC, in a subject, the method further comprises counseling the subject with increased susceptibility to developing cancer on prevention of cancer. In some cases, counseling the subject with increased susceptibility to developing cancer comprises advising the subject to reduce alcohol consumption and/or use of tobacco products. Counseling the subject with increased susceptibility to developing cancer can also include advising the subject to increase dietary intake of fruits, vegetables, olive oil and/or fish oils, and/or reduce dietary intake of red meat, fried food and/or fat; and/or advising the subject to obtain frequent screening (such as oropharyngeal and/or nasopharyngeal examination for HNSCC).

In some embodiments of the diagnostic method, the method further includes treating the subject for SCC. Any appropriate treatment can be used for treating the SCC. The treatment method selected will depend on a variety of factors, including for example, the type and location of the SCC, the stage of disease, and overall health of the subject. In some examples, the treatment is selected from administering a therapeutically effective amount of an inhibitor of the PI3K/Akt pathway; administering a therapeutically effective amount of a modulator of the TGF-β pathway; surgical removal of the SCC tumor; administering radiation therapy; administering chemotherapy; or any combination thereof.

Also provided herein is a method of treating a subject with cancer, such as SCC, by selecting a subject in need of treatment; and administering to the subject a therapeutically effective amount of a treatment (such as radiation therapy, chemotherapy, surgery or any type of anti-cancer treatment) for the SCC. In some embodiments, the anti-cancer treatment is an inhibitor of the PI3K/Akt pathway and a therapeutically effective amount of a modulator of the TGF-β pathway. Administration of the treatment (such as the inhibitor of the PI3K/Akt pathway and modulator of the TGF-β pathway) results in reduction in tumor size, inhibition of tumor growth, inhibition of tumor metastasis or inhibition of tumor progression, thereby treating the subject diagnosed with cancer. In some embodiments, the cancer is a SCC. In some examples, the SCC is a HNSCC. In some examples, the SCC is a SCC of the skin, oral mucosa, tongue, penis, vagina, cervix, peri-orbital region or peri-anal region. In some embodiments, the cancer is an adenocarcinoma, such as prostate cancer.

In some embodiments, the inhibitor of the PI3K/Akt pathway is an inhibitor of PI3K, AKT, pyruvate dehydrogenase kinase (PDK1) or mammalian target of rapamycin (mTOR). The inhibitor of the PI3K/Akt pathway can be any type of compound that inhibits expression or activity of a member of the PI3K/Akt pathway. In some embodiments, the inhibitor is a small molecule, antibody, antisense compound or polypeptide. In some examples, the antibody is a chimeric antibody, a humanized antibody or a human antibody. In some examples, the antisense compound is an antisense oligonucleotide, siRNA, miRNA, shRNA or ribozyme. Antibodies, antisense compounds and other inhibitors specific for members of the PI3K/Akt pathway are known in the art and are commercially available. Exemplary inhibitors of the PI3K/Akt pathway are described herein, but are not intended to be limiting.

In some embodiments of the methods, the modulator of the TGF-β pathway is an inhibitor of the TGF-β pathway. In some cases, the inhibitor is a small molecule, antibody, antisense compound or polypeptide. In some examples, the antibody is a chimeric antibody, a humanized antibody or a human antibody. In some examples, the antisense compound is an antisense oligonucleotide, siRNA, miRNA, shRNA or ribozyme. In some examples, the polypeptide is a fusion protein. Antibodies, antisense compounds and other modulators specific for members of the TGF-β pathway are known in the art and are commercially available. Exemplary inhibitors of the TGF-β pathway are described herein, but are not intended to be limiting.

In some embodiments, the modulator of the TGF-β pathway is an activator of the TGF-β pathway. In some examples, the TGF-β pathway activator is a TGF-β mimetic (see, for example, Glaser et al., Mol. Cancer. Ther. 1:759-768, 2002), an isoprenoid (see, for example, Lee et al., Am. J. Respir. Cell Mol. Biol. 31:234-240, 2004), RAP250 (see, for example, Antonson et al., J. Biol. Chem. 283(14):8995-9001, 2008) or LM04 (Lu et al., Oncogene 25:2920-2930, 2006). Other TGF-β pathway activators are known in the art and can be used in the disclosed methods (see, for example, Sponer et al., J. Cataract Surg. 31:595-606k 2005; Wahab et al., Exp. Cell Res. 307:305-314, 2005).

In some embodiments, the modulator of the TGF-β pathway is a modulator of TGF-β or TGF-β receptor. In some examples, the TGF-β is TGF-β₁, TGF-β₂ or TGF-β₃. In some examples, the TGF-β receptor is TGFBR1 or TGFBR2.

Inhibitors and modulators can be administered to a subject using a suitable route of administration. In some embodiments, the route of administration is oral, topical or systemic.

Further provided are pharmaceutical compositions comprising an inhibitor of the PI3K/Akt pathway and a modulator of the TGF-β pathway. In some embodiments, the modulator of the TGF-β pathway is an inhibitor of the TGF-β pathway. In other embodiments, the modulator of the TGF-β pathway is an activator of the TGF-β pathway. In some embodiments, the modulator of the TGF-β pathway is a modulator of TGF-β or TGF-β receptor. In some examples, the TGF-β is TGF-β₁, TGF-β₂ or TGF-β₃. In some examples, the TGF-β receptor is TGFBR1 or TGFBR2.

In some embodiments of the disclosed pharmaceutical compositions, the inhibitor or modulator is a small molecule, antibody or antisense compound. In some examples, the antibody is a chimeric antibody, a humanized antibody or a human antibody. In some examples, the antisense compound is an antisense oligonucleotide, siRNA, miRNA, shRNA or ribozyme. In some embodiments, the pharmaceutical compositions further comprise a pharmaceutically acceptable carrier. Also provided is the use of the pharmaceutical compositions disclosed herein for the preparation of a medicament for the treatment of cancer, particularly SCC. In some examples, the SCC is a HNSCC. In some examples, the SCC is a SCC of the skin, oral mucosa, tongue, peri-orbital region, penis, vagina, cervix or peri-anal region. In some embodiments, the cancer is an adenocarcinoma, such as prostate cancer.

Also provided herein is a genetically modified non-human animal comprising a homozygous deletion of the TGFBR1 gene and a homozygous deletion of the PTEN gene, wherein the non-human animal is highly susceptible to developing SCC tumors, such as HNSCC tumors. In some embodiments, the non-human animal is a mouse. In some embodiments, the deletion of the TGFBR1 gene and the deletion of the PTEN gene are conditional deletions. In some embodiments, the deletions occur only in the head and neck epithelia of the non-human animal. In a particular example disclosed herein, conditional deletion of TGFBR1 and PTEN occur following exposure of a genetically modified mouse to tamoxifen, which drives expression of Cre recombinase, resulting in conditional deletion of TGFBR1 and PTEN. Methods of generating genetically modified animals are well known in the art and are described in greater detail below.

A method of screening therapeutic agents useful for the treatment of SCC is also provided herein. The screening method comprises (i) providing a genetically modified non-human animal with a homozygous deletion of the TGFBR1 gene and a homozygous deletion of the PTEN gene; (ii) administering a candidate therapeutic agent to the genetically modified animal; and (iii) determining the effect of administering the candidate therapeutic agent to the genetically modified animal. A reduction in tumor size, inhibition of tumor growth, inhibition of tumor metastasis or inhibition of tumor progression in the genetically modified animal identifies the candidate agent as a therapeutic agent useful for the treatment of SCC. Candidate therapeutic agents can be any type of compound, such as an antibody, polypeptide, polynucleotide, small molecule or antisense compound. In some embodiments, the SCC is HNSCC.

### IV. Role of the TGF-β Pathway in Promotion of Tumor Initiation and Progression

TGF-β is a potent growth inhibitor for epithelial cells (Massagué and Gomis, FEBS Lett 580:2811-2820, 2006) and plays an important role in HNSCC development. Particularly, TGF-β inhibits proliferation of head and neck epithelia at an early stage (Xie et al., Oncol Res 14:61-73, 2003). However, the precise role of TGF-β signaling in head and neck carcinogenesis has not been fully understood. Existing research has been mainly focused on TGFBR2. Previous reports have revealed that the expression of the dominant negative type II receptor (ΔβRII) increased susceptibility to chemical carcinogenesis protocols at both early and late stages. It also decreased the latency of mammary tumor formation while significantly reducing the incidence of extravascular lung metastasis (Siegel et al., Proc Natl Acad Sci USA 100:8430-8435, 2003). Inactivation of Tgfbr2 in colon epithelial cells did not cause spontaneous neoplasm formation but promoted the establishment and progression of colon neoplasms after a concurrent initiating event (Biswas et al., Cancer Res 64:4687-4692, 2004). Mice with a targeted deletion of Tgfbr2 in head and neck epithelia had no significant pathological changes. Only K-ras^{12D/+}/Tgfbr2^{-/-} transgenic mice or DMBA-initiated Tgfbr2^{-/-} mice developed HNSCCs (Lu et al., Genes Dev 20:1331-1342, 2006). In addition to TGF-β autocrine effects on tumor progression, the excess production of TGF-β by cancer cells can also contribute to cancer development through paracrine mechanisms. Mice that harbored an inactivated Tgfbr2 in stromal cells developed intraepithelial neoplasia of the prostate and invasive SCCs in the forestomach, suggesting that alterations in the TGF-β signaling pathway within the tumor microenvironment also contribute to cancer development and progression (Bhowmick et al., Science 303:848-851, 2004).

However, TGFBR2 interacts not only with TGFBR1, but also forms functional complexes with other type I receptors such as ActRI/ALK2 or ALK1 (Feng and Derynck, Annu Rev Cell Dev Biol 21:659-693, 2005). The later complexes signal through Smad1, Smad5, and Smad8, which is different from that involving TGFBR1, which results in phosphorylation of Smad2 and Smad3. In fact, TGF-β signaling through TGFBR1 and ALK1, in a complex with TGFBR2, showed opposing activities in endothelial cell migration and proliferation (Goumans et al., EMBO J. 21:1743-1753, 2002). In epithelial cells, TGFBR2 can also directly phosphorylate Par6 without involvement of TGFBR1, and release it from the Par6-TGFBR1 complex, which allows Par6 to trigger the dissolution of tight junctions in the context of epithelial-mesenchymal transitions (Ozdamar et al., Science 307:1603-1609, 2005). Therefore, knocking out Tgfbr2 affects not only Smad2/3- and Smad1/5/8-mediated TGF-β signaling but also direct receptor-II-mediated alternative signaling via Par6. This makes it difficult to study the specificity of Smad-mediated signaling, which plays a crucial role in tumor progression.

TGFBR1 is a member of the TGF-β family of receptors, which is active in both Smad-dependent and Smad-independent pathways. It forms heterotetrameric complexes with TGFBR2 on the cell surface and serves as a specific receptor for TGF-βs. Despite its structural similarity with the other TGF-β receptors, its precise role in HNSCC is not clearly defined. Mutations and polymorphisms of TGFBR1 have been described. TGFBR1 (6A), a 9 bp deletion coding for 3 alanine residues within the 9 alanine repeat region of exon 1 has been particularly associated with HNSCC (Chen et al., Int J Cancer 93:653-61, 2001; Knobloch et al., Mutat Res 479:131-9, 2001; Pasche et al., JAMA 294:1634-46, 2005). Furthermore, previous studies have shown that 35% of mice with a targeted deletion of Tgfbr1 developed spontaneous SCCs in periorbital and/or perianal regions (Honjo et al., Cell Cycle 6:1360-1366, 2007). Thus, in some circumstances, TGFBR1 might function independently of TGFBR2 and exert additional effects in cancer development. To study the role of Tgfbr1 signaling in head and neck cancer, a novel inducible knockout model was developed by crossing Tgfbr1 floxed mice with Kl4-CreER^{tam} mice. By deleting Tgfbr1 in head and neck epithelia, it was possible to identify more specifically the role of this receptor and its direct downstream target proteins, Smad2 and Smad3, in the progression of HNSCCs. With these DMBA-induced Tgfbr1 cKO mice, it is possible to model an aspect of HNSCC that is rare in existing models.

The results from the Tgfbr1 cKO mouse model disclosed herein indicate that targeted deletion of Tgfbr1 alone in head and neck epithelia is not sufficient to develop spontaneous tumor in these mice. Instead, loss of Tgfbr1 promotes head and neck carcinogenesis in mice in combination with DMBA treatment. Most of the findings on Tgfbr1 cKO mouse model disclosed herein are consistent with the findings from DMBA-initiated Tgfbr2 cKO mice (Lu et al., Genes Dev 20:1331-1342, 2006), suggesting that Tgfbr1 functions similar to Tgfbr2 in the progression of HNSCCs. The lack of spontaneous tumor formation in Tgfbr1 cKO mice, together with the fact that DMBA treatment facilitates tumor development in these mice suggests that rather than initiation, loss of Tgfbr1 may play a more crucial role in tumor progression in mouse HNSCC. However, several differences were noted in the DMBA-initiated Tgfbr1 cKO mice compared with DMBA-initiated Tgfbr2 cKO mice. For example, none of the DMBA-initiated Tgfbr1 heterozygous mice (Tgfbr1^{+/-}) developed HNSCCs, while about 33% of mice with a heterozygous Tgfbr2 deletion in the head and neck epithelia (Tgfbr2^{+/-}) developed HNSCCs after DMBA initiation. Furthermore, only 16% of the DMBA-initiated Tgfbr1 cKO mice with tumors developed metastases in jugular lymph nodes and/or lungs by the time the mice were dissected. However, up to 35% of the DMBA-initiated Tgfbr2 cKO mice developed jugular lymph node metastases by 20-39 weeks of age. The differences between these two mouse models indicate that Tgfbr1 and Tgfbr2 function differently, with Tgfbr2 having more suppressive effects in later stages of cancer development, possibly due to TGFBR1-independent effects.

It is widely believed that TGF-β causes cancer progression through both autocrine and paracrine effects. Paracrine effects of TGF-β include stimulation of inflammation and angiogenesis, escape from immunosurveillance, and recruitment of myofibroblasts, while autocrine effects of TGF-β in cancer cells with a functional TGF-β receptor complex may be caused by a convergence of TGF-β signaling with other signaling pathways (De Wever and Mareel, J Pathol 200:429-447, 2003). In the studies disclosed herein, it was found that upon deletion of Tgfbr1 in mouse head and neck epithelia, there is an enhanced cell proliferation and down-regulation of cell cycle inhibitors due to inactivation of Smad2/3 mediated signaling. An inhibition of apoptosis through activation of PI3K/Akt pathway in SCCs that developed in Tgfbr1 cKO mice was also observed. These results suggest that in the head and neck epithelia, TGF-β is an early tumor suppressor. In the SCCs that developed in Tgfbr1 cKO mice, increased inflammation, angiogenesis, and myofibroblast formation were found. Similar results have been observed in other mouse models when TGF-β signaling was disrupted either by tissue-specific transgenic expression of a dominant negative Tgfbr2 (ΔβRII) in the epidermis (Go et al., Cancer Res 59:2861-2868, 1999) or through targeted deletion of Tgfbr2 in mouse head and neck epithelia (Lu et al., Genes Dev 20:1331-1342, 2006). Furthermore, elevated levels of endogenous TGF-β1 were detected in tumor stroma in Tgfbr1 cKO mice as it has been seen in other studies (Lu et al., Genes Dev 20:1331-1342, 2006). The deletion of Tgfbr1 in mouse head and neck epithelia prevents the surrounding increased TGF-β1 from exerting their tumor suppressive effects. However, expression of Tgfbr1 in tumor stroma enhances its tumor promoting function through paracrine effects. Therefore, despite the fact that inflammation induces angiogenesis and tumorigenesis, it is believed that the elevated level of TGF-β1 in tumor stroma has direct involvement in creating microenvironment for tumor progression (Lu et al., Cancer Res 64:4405-4410, 2004).

Alternative modes of TGF-β signaling have been categorized into 3 groups: Smad4-independent RSmad signaling (via interactions with TIF1γ, IKKα, and p68DROSHA), Smad-independent receptor-1 signaling (via small G proteins and MAPK pathways), and direct receptor-II signaling (via Par6, and via LIMK in the case of BMPR-II) (Massagué, Cell 59:3379-3386, 1999). Recent work showed that TGF-β induces apoptosis through repression of PI3K/Akt signaling, indicating that there may be negative crosstalk between the TGF-β tumor suppressor and PI3K/Akt pathways (Wang et al., Cancer Res 68:3152-3160, 2008). One of the most notable findings of the current study is that after deletion of Tgfbr1 in mouse head and neck epithelia and DMBA treatment, in addition to inactivation of the Smad-dependent TGF-β signaling pathway, one of the most important Smad-independent receptor-1 pathways, the PI3K/Akt pathway is activated in SCCs that developed in the Tgfbr1 cKO mice. The results from the studies disclosed herein indicate that decreased Tgfbr1 expression in Tgfbr1 cKO mice leads to increased cell proliferation and cell survival through PTEN independent activation of PI3K/Akt pathway, possibly due to DMBA induced H-ras mutation as well as other unknown mechanisms. These changes accompanied by increased TGF-β1 in tumor stroma, which leads to increased invasion, angiogenesis, inflammation as well as immune suppression through paracrine effect of TGF-β, switches TGF-β signaling from tumor suppression in normal cells to tumor promotion in head and neck carcinogenesis of Tgfbr1 cKO mice. These results indicate that the inactivation of TGF-β signaling, in the context of ras mutations and aberrant activation of the PI3K/Akt pathway, may contribute cooperatively to promote head and neck carcinogenesis in these mice. These findings reveal a critical role of the TGF-β signaling pathway and its crosstalk with PI3K/Akt pathway in suppressing head and neck carcinogenesis.

### V. Modulators of the TGF-β and PI3K/Akt Pathways

A. Small Molecule Inhibitors

A number of small molecule inhibitors that modulate activity of members of the TGF-β or PI3K/Akt pathways have been previously described. Any known, or yet to be described small molecule that inhibits activity of one or more members of the TGF-β or PI3K/Akt pathway is contemplated for use in the disclosed methods. Methods of identifying small molecule inhibitors to a specific molecule are within the abilities of one of skill in the art.

i. TGF-β Pathway

As described herein, an inhibitor of the TGF-β pathway is any compound that directly or indirectly inhibits expression or activity of a member of the TGF-β pathway. In some embodiments, the inhibitor of the TGF-β pathway is a TGF-β inhibitor. An inhibitor of TGF-β can inhibit all isoforms (TGF-β₁, TGF-β₂, TGF-β₃), or a single isoform. In some embodiments, the inhibitor of the TGF-β pathway is a TGF-β receptor inhibitor. An inhibitor of TGF-β receptor can inhibit both types (TGF-β receptor type I or type II), or a single type.

In some examples, the TGF-β receptor small molecule inhibitor is SB-431542 (inhibitor of TGF-β receptor II; 4-(5-Benzol[1,3]dioxol-5-yl-4-pyrldin-2-yl-1H-imidazol-2-yl)-benzamide hydrate, 4-[4-(3,4-Methylenedioxyphenyl)-5-(2-pyridyl)-1H-imidazol-2-yl]-benzamide hydrate, 4-[4-(1,3-Benzodioxol-5-yl)-5-(2-pyridinyl)-1H-imidazol-2-yl]-benzamide hydrate); NPC-30345 (small molecule inhibitor of TGF-β receptor I; Scios Inc., Fremont, Calif.); LY364947 (small molecule inhibitor of TGF-β receptor I; 4-[3-(2-Pyridinyl)-1H-pyrazol-4-yl]-quinoline); A-83-01 (inhibitor of TGF-β receptor type I; 3-(6-Methylpyridin-2-yl)-1-phenylthiocarbamoyl-4-quinolin-4-ylpyrazole); LY550410 (inhibitor of TGF-β receptor type I; Lilly Research); LY580276 (inhibitor of TGF-β receptor type I; Lilly Research); LY566578 (inhibitor of TGF-β receptor type I; Lilly Research); SB-505124 (selective inhibitor of TGF-β receptor type I; 2-(5-benzo[1,3]dioxol-5-yl-2-tert-butyl-3H-imidazol-4-yl)-6-methylpyridine hydrochloride); SD-093 (inhibitor of TGF-β receptor type I; Scios Inc.); or SD-208 (inhibitor of TGF-β receptor type I; Scios Inc.). The above small molecule inhibitors have been described in the art (see, for example, DaCosta Byfield et al., Mol. Pharmacol. 65(3):744-752, 2004; Ge et al., Biochem Pharmacol. 68(1):41-50, 2004; Inman et al., Mol. Pharmacol. 62(1):65-74, 2002; Tojo et al., Cancer Sci. 96(11):791-800, 2005; Uhl et al., Cancer Res. 64(21):7954-7961, 2004; Sawyer et al., BioMed Chem Lett 14:3581-3584; Hjelmeland et al., Mol Cancer Ther 3:737-745, 2003).

ii. PI3K/Akt Pathway

As described herein, an inhibitor of the PI3K/Akt pathway is any compound that directly or indirectly inhibits expression or activity of a member of the PI3K/Akt pathway. In some embodiments, the inhibitor of the PI3K/Akt pathway is an inhibitor of PI3K, Akt, pyruvate dehydrogenase kinase (PDK1) or mammalian target of rapamycin (mTOR). In some examples, the small molecule inhibitor of PI3K is LY294002 (also known as 2-(4-morpholinyl)-8-phenyl-4H-1-benzopyran-4-one; molecular formula C₁₉H₁₇NO₃) or wortmannin (molecular formula C₂₃H₂₄O₈). In some examples, the small molecule inhibitor of Akt is UCN-01 (also known as 7-hydroxystaurosporine and 8,12-epoxy-1H,8H-2,7b,12a-triazadibenzo[a,g]cyclonona[cde]trinden-1-one, 2,3,9,10,11,12-hexahydro-3-hydroxy-9-methoxy-8-methyl-10-(methylamino)). UCN-01 is a synthetic derivative of staurosporine with antineoplastic activity.

As described herein, an mTOR inhibitor is a molecule that inhibits expression or activity of mTOR. For example, an mTOR inhibitor can be a molecule that inhibits the kinase activity of mTOR or inhibits binding of mTOR to a ligand. In some embodiments, the mTOR inhibitor is rapamycin or a rapamycin analog. Rapamycin is a small molecule with known immunosuppressive and anti-proliferative properties. Rapamycin, also known as sirolimus, is a macrolide that was first discovered as a product of the bacterium Streptomyces hygroscopicus. Rapamycin binds and inhibits the activity of mTOR. The chemical formula of rapamycin is C₅₁H₇₉NO₁₃ and the International Union of Pure and Applied Chemistry (IUPAC) name is (3S,6R,7E,9R,10R,12R,14S,15E,17E,19E,21S,23S,26R,27R,34aS)-9,10,12,13,14,21,22,23,24,25,26,27,32,33,34,34a-hexadecahydro-9,27-dihydroxy-3-[(1R)-2-[(1S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4]-oxaazacyclohentriacontine-1,5,11,28,29(4H,6H,31H)-pentone. Analogs of rapamycin are known and include, for example, CCI-779 (also called temsirolimus and Torisel™) and RAD-001 (also known as 42-O-(2-hydroxy)ethyl rapamycin and everolimus).

B. Antisense Compounds

Generally, the principle behind antisense technology is that an antisense compound hybridizes to a target nucleic acid and effects the modulation of gene expression activity, or function, such as transcription, translation or splicing. The modulation of gene expression can be achieved by, for example, target RNA degradation or occupancy-based inhibition. An example of modulation of target RNA function by degradation is RNase H-based degradation of the target RNA upon hybridization with a DNA-like antisense compound, such as an antisense oligonucleotide. Antisense oligonucleotides can also be used to modulate gene expression, such as splicing, by occupancy-based inhibition, such as by blocking access to splice sites.

Another example of modulation of gene expression by target degradation is RNA interference (RNAi) using small interfering RNAs (siRNAs). RNAi is a form of antisense-mediated gene silencing involving the introduction of double stranded (ds) RNA-like oligonucleotides leading to the sequence-specific reduction of targeted endogenous mRNA levels. Another type of antisense compound that utilizes the RNAi pathway is microRNA. MicroRNAs are naturally occurring RNAs involved in the regulation of gene expression. However, these compounds can be synthesized to regulate gene expression via the RNAi pathway. Similarly, shRNAs are RNA molecules that form a tight hairpin turn and can be used to silence gene expression via the RNAi pathway. The shRNA hairpin structure is cleaved by the cellular machinery into siRNA.

Other compounds that are often classified as antisense compounds are ribozymes. Ribozymes are catalytic RNA molecules that can bind to specific sites on other RNA molecules and catalyze the hydrolysis of phosphodiester bonds in the RNA molecules. Ribozymes modulate gene expression by direct cleavage of a target nucleic acid, such as a messenger RNA.

Each of the above-described antisense compounds provides sequence-specific target gene regulation. This sequence-specificity makes antisense compounds effective tools for the selective modulation of a target nucleic acid of interest. In some embodiments provided herein, the target nucleic acid molecule is a nucleic acid molecule encoding a member of the TGF-β pathway or encoding a member of the PI3K/Akt pathway.

i. TGF-β and PI3K/AKT Pathway Antisense Compounds

As taught herein, dual modulation of the TGF-β pathway and PI3K/Akt pathway can be used to treat HNSCC. Accordingly, provided herein is a method of treating cancer, such as SCC, in a subject by administering a modulator of a member of the TGF-β pathway and an inhibitor of the PI3K/Akt pathway. Members of the TGF-β and PI3K/Akt pathways are known in the art and are described herein. Nucleic acid sequences for members of the TGF-β and PI3K/Akt pathways are publically available. Based on known nucleic acid sequences, one is capable of designing antisense compounds specific for a target of interest, as described in greater detail below.

Antisense compounds specific for members for the TGF-β pathway have been previously described. For example, AP-11014 and AP-12009 (Antisense Pharma, Regensburg, Germany) are antisense oligonucleotides specific for TGF-β₁ and TGF-β₂, respectively (Schlingensiepen et al., Cytokine Growth Factor Rev. 17:129-139, 2006; Schlingensiepen et al., Am Soc Clin Oncol Ann Meeting Abstract 3132, 2004; Bogdahn et al., Am Soc Clin Oncol Ann Meeting Abstract 1514, 2004). Other antisense oligonucleotides specific for one or more isoforms of TGF-β are described in, for example, U.S. Pat. Nos. 6,884,787; 6,455,689; and 6,841,542; and U.S. Patent Application Publication Nos. 2008/0214483; 2004/0063655; 2004/0006030; 2003/0153075; 2003/0050265; and 2003/0078217. TGF-β receptor antisense oligonucleotides are disclosed in, for example, U.S. Patent Application Publication Nos. 2004/0147472; and 2003/0064944. TGF-β- and TGF-β receptor-specific siRNA molecules are disclosed in, for example, U.S. Patent Application Publication Nos. 2005/0287128; and 2005/0227936.

Antisense compounds specific for members for the PI3K/Akt pathway have been previously described. For example, U.S. Patent Application Publication Nos. 2005/02772682 and 2004/0077580 disclose siRNAs and antisense oligonucleotides specific for PI3K. In addition, U.S. Patent Application Publication Nos. 2008/0161547, 2004/0265999 and 2003/0148974 describe antisense oligonucleotide and siRNA compounds that target AKT. PCT Publication No. WO 2000/061786 discloses PDK-1 specific antisense compounds.

In some embodiments, expression of the TGF-β or PI3K/Akt pathway member is inhibited at least about 10%, at least about 25%, at least 50%, at least 75%, at least 90%, or at least 95% relative to a control (such as compared to an untreated subject, or expression prior to treatment). Any type of antisense compound that specifically targets and regulates expression of a TGF-β or PI3K/Akt pathway member is contemplated for use with the disclosed methods. Such antisense compounds include single-stranded compounds, such as antisense oligonucleotides, and double-stranded compounds, including compounds with at least partial double-stranded structure, including siRNAs, miRNAs, shRNAs and ribozymes. Methods of designing, preparing and using antisense compounds that specifically target a nucleic acid molecule encoding a TGF-β or PI3K/Akt pathway member are within the abilities of one of skill in the art. Furthermore, sequences for TGF-β and PI3K/Akt pathway members are publicly available (see Terms and Methods for exemplary GenBank Accession Numbers, which are herein incorporated by reference as they appear in the GenBank database as of Apr. 24, 2009). The specific GenBank Accession numbers listed herein are provided for reference only and are not intended to be limiting.

Antisense compounds specifically targeting a TGF-β or PI3K/Akt pathway member nucleic acid molecule can be prepared by designing compounds that are complementary to the TGF-β or PI3K/Akt pathway member nucleotide sequence, particularly the TGF-β or PI3K/Akt pathway member mRNA sequence. Antisense compounds targeting a TGF-β or PI3K/Akt pathway member need not be 100% complementary to the TGF-β or PI3K/Akt pathway member to specifically hybridize and regulate expression the target gene. For example, the antisense compound, or antisense strand of the compound if a double-stranded compound, can be at least 75%, at least 80%, at least 85%, at least 90%, at least 95%, at least 99% or 100% complementary to the selected TGF-β or PI3K/Akt pathway member nucleic acid sequence. Methods of screening antisense compounds for specificity are well known in the art (see, for example, U.S. Patent Application Publication No. 2003/0228689).

ii. Antisense Compound Modifications

In some examples, the antisense compounds described herein contain one or more modifications to enhance nuclease resistance and/or increase activity of the compound. Modified antisense compounds include those comprising modified backbones or non-natural internucleoside linkages. As defined herein, antisense compounds having modified backbones include those that retain a phosphorus atom in the backbone and those that do not have a phosphorus atom in the backbone.

Examples of modified oligonucleotide backbones include, but are not limited to, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkyl-phosphonates, thionoalkylphosphotriesters, and boranophosphates having normal 3′-5′ linkages, 2′-5′ linked analogs of these, and those having inverted polarity wherein the adjacent pairs of the nucleoside units are linked 3′-5′ to 5′-3′ or 2′-5′ to 5′-2′. Representative U.S. patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Examples of modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

In some embodiments, both the sugar and the internucleoside linkage of the nucleotide units of the antisense compound are replaced with novel groups. One such modified compound is an oligonucleotide mimetic referred to as a peptide nucleic acid (PNA). In PNA compounds, the sugar-backbone of an oligonucleotide is replaced with an amide containing backbone, in particular an aminoethylglycine backbone. The bases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262. Further teaching of PNA compounds can be found in Nielsen et al. (Science 254, 1497-1500, 1991).

Modified antisense compounds can also contain one or more substituted sugar moieties. In some examples, the antisense compounds can comprise one of the following at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. In other embodiments, the antisense compounds comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In one example, the modification includes 2′-methoxyethoxy (also known as 2′-O-(2-methoxyethyl) or 2′-MOE) (Martin et al., Helv. Chim. Acta. 78, 486-504, 1995). In other examples, the modification includes 2′-dimethylaminooxyethoxy (also known as 2′-DMAOE) or 2′-dimethylaminoethoxyethoxy (also known in the art as 2′-O-dimethylaminoethoxyethyl or 2′-DMAEOE).

Similar modifications can also be made at other positions of the compound. Antisense compounds can also have sugar mimetics such as cyclobutyl moieties in place of the pentofuranosyl sugar. Representative United States patents that teach the preparation of modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920.

Antisense compounds can also include base modifications or substitutions. As used herein, “unmodified” or “natural” bases include the purine bases adenine (A) and guanine (G), and the pyrimidine bases thymine (T), cytosine (C) and uracil (U). Modified bases include other synthetic and natural bases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further modified bases have been described (see, for example, U.S. Pat. No. 3,687,808; and Sanghvi, Y. S., Chapter 15, Antisense Research and Applications, pages 289-302, Crooke, S. T. and Lebleu, B., ed., CRC Press, 1993). Certain of these modified bases are useful for increasing the binding affinity of antisense compounds. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. Representative U.S. patents that teach the preparation of modified bases include, but are not limited to, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,750,692.

C. Antibodies

Antibodies contemplated for use in the methods provided herein include, for example, monoclonal and polyclonal antibodies specific for a protein, or fragment thereof, of the TGF-β or PI3K/Akt pathway.

i. TGF-β Pathway Antibodies

In some embodiments, the antibody specific for a member of the TGF-β pathway is an antibody specific for TGF-13. An antibody specific for TGF-β can target all isoforms (TGF-β₁, TGF-β₂, TGF-β₃), or a single isoform. In some examples, the TGF-β inhibitor is CAT-192, a monoclonal antibody specific for human TGF-β₁ (U.S. Pat. No. 6,492,497; Cordeiro et al., Invest Ophthalmol Vis Sci 40(10):2225-2234, 1999; Benigni et al., J Am Soc Nephrol 14:1816-1824, 2003); CAT-152, a monoclonal antibody specific for human TGF-β₂ (PCT Publication No. WO 97/13844; Thompson et al., J Immunol Methods 227(1-2):17-29, 1999; Mead et al., Invest Ophthalmol Vis Sci 44:3394-3401, 2003); 1D11, a monoclonal antibody that inhibits TGF-β₁ and TGF-β₂ (U.S. Patent Application Publication No. 2002/0176858; U.S. Pat. No. 6,090,383; Dasch et al., J Immunol 142(5):1536-1541, 1989); or 2G7, a pan-TGF-β monoclonal antibody (Lucas et al., J Immunol 145(5):1415-22, 1990).

ii. PI3K/Akt Pathway Antibodies

Antibodies specific for members of the PI3K/Akt pathway have been described in the art and are commercially available from a variety of sources. For example, PI3K antibodies are disclosed in U.S. Patent Application Publication No. 2008/0014598.

iii. Methods of Making Polyclonal and Monoclonal Antibodies

Methods of making polyclonal and monoclonal antibodies are well known, and are described below. Polyclonal antibodies, antibodies which consist essentially of pooled monoclonal antibodies with different epitopic specificities, as well as distinct monoclonal antibody preparations are included. The preparation of polyclonal antibodies is well known to those skilled in the art (see, for example, Green et al., “Production of Polyclonal Antisera,” in: Immunochemical Protocols, pages 1-5, Manson, ed., Humana Press, 1992; Coligan et al., “Production of Polyclonal Antisera in Rabbits, Rats, Mice and Hamsters,” in: Current Protocols in Immunology, section 2.4.1, 1992).

The preparation of monoclonal antibodies likewise is conventional (see, for example, Kohler & Milstein, Nature 256:495, 1975; Coligan et al., sections 2.5.1-2.6.7; and Harlow et al. in: Antibodies: a Laboratory Manual, page 726, Cold Spring Harbor Pub., 1988). Briefly, monoclonal antibodies can be obtained by injecting mice with a composition comprising an antigen, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (see, e.g., Coligan et al., sections 2.7.1-2.7.12 and sections 2.9.1-2.9.3; Barnes et al., Purification of Immunoglobulin G (IgG), in: Methods in Molecular Biology, Vol. 10, pages 79-104, Humana Press, 1992).

Methods of in vitro and in vivo multiplication of monoclonal antibodies are well known to those skilled in the art. Multiplication in vitro may be carried out in suitable culture media such as Dulbecco's Modified Eagle Medium or RPMI 1640 medium, optionally supplemented by a mammalian serum such as fetal calf serum or trace elements and growth-sustaining supplements such as normal mouse peritoneal exudate cells, spleen cells, thymocytes or bone marrow macrophages. Production in vitro provides relatively pure antibody preparations and allows scale-up to yield large amounts of the desired antibodies. Large-scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells, such as syngeneic mice, to cause growth of antibody-producing tumors. Optionally, the animals are primed with a hydrocarbon, especially oils such as pristane (tetramethylpentadecane) prior to injection. After one to three weeks, the desired monoclonal antibody is recovered from the body fluid of the animal.

Antibodies can also be derived from a subhuman primate antibody. General techniques for raising therapeutically useful antibodies in baboons can be found, for example, in PCT Publication No. WO 91/11465; and Losman et al., Int. J. Cancer 46:310, 1990.

Alternatively, an antibody that specifically binds a TGF-β or PI3K/Akt pathway member polypeptide can be derived from a humanized monoclonal antibody. Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Natl. Acad. Sci. U.S.A. 86:3833, 1989. Techniques for producing humanized monoclonal antibodies are described, for example, by Jones et al., Nature 321:522, 1986; Riechmann et al., Nature 332:323, 1988; Verhoeyen et al., Science 239:1534, 1988; Carter et al., Proc. Natl. Acad. Sci. U.S.A. 89:4285, 1992; Sandhu, Crit. Rev. Biotech. 12:437, 1992; and Singer et al., J. Immunol. 150:2844, 1993.

Antibodies can be derived from human antibody fragments isolated from a combinatorial immunoglobulin library. See, for example, Barbas et al., in: Methods: a Companion to Methods in Enzymology, Vol. 2, page 119, 1991; Winter et al., Ann. Rev. Immunol. 12:433, 1994. Cloning and expression vectors that are useful for producing a human immunoglobulin phage library can be obtained, for example, from Stratagene Cloning Systems (La Jolla, Calif.).

In addition, antibodies can be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13, 1994; Lonberg et al., Nature 368:856, 1994; and Taylor et al., Int. Immunol. 6:579, 1994.

Antibodies include intact molecules as well as fragments thereof, such as Fab, F(ab′)₂, and Fv which are capable of binding the epitopic determinant. These antibody fragments retain some ability to selectively bind with their antigen or receptor and are defined as follows:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody, defined as a genetically engineered molecule containing the variable region of the light chain, the variable region of the heavy chain, linked by a suitable polypeptide linker as a genetically fused single chain molecule.

Methods of making these fragments are known in the art (see for example, Harlow and Lane, Antibodies: A Laboratory Manual, Cold Spring Harbor Laboratory, New York, 1988). An epitope is any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

Antibody fragments can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. No. 4,036,945 and U.S. Pat. No. 4,331,647, and references contained therein; Nisonhoff et al., Arch. Biochem. Biophys. 89:230, 1960; Porter, Biochem. J. 73:119, 1959; Edelman et al., Methods in Enzymology, Vol. 1, page 422, Academic Press, 1967; and Coligan et al. at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. For example, Fv fragments comprise an association of V_{H} and V_{L} chains.

This association may be noncovalent (Inbar et al., Proc. Natl. Acad. Sci. U.S.A. 69:2659, 1972). Alternatively, the variable chains can be linked by an intermolecular disulfide bond or cross-linked by chemicals such as glutaraldehyde (see, for example, Sandhu, Crit. Rev. Biotech. 12:437, 1992). Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are known in the art (see Whitlow et al., Methods: a Companion to Methods in Enzymology, Vol. 2, page 97, 1991; Bird et al., Science 242:423, 1988; U.S. Pat. No. 4,946,778; Pack et al., Bio/Technology 11:1271, 1993; and Sandhu, Crit. Rev. Biotech. 12:437, 1992).

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (Larrick et al., Methods: a Companion to Methods in Enzymology, Vol. 2, page 106, 1991).

Antibodies can be prepared using an intact polypeptide or fragments containing small peptides of interest as the immunizing antigen. The polypeptide or a peptide used to immunize an animal can be derived from substantially purified polypeptide produced in host cells, in vitro translated cDNA, or chemical synthesis which can be conjugated to a carrier protein, if desired. Such commonly used carriers which are chemically coupled to the peptide include keyhole limpet hemocyanin, thyroglobulin, bovine serum albumin, and tetanus toxoid. The coupled peptide is then used to immunize the animal (e.g., a mouse, a rat, or a rabbit).

Polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (see, for example, Coligan et al., Unit 9, Current Protocols in Immunology, Wiley Interscience, 1991).

Binding affinity for a target antigen is typically measured or determined by standard antibody-antigen assays, such as competitive assays, saturation assays, or immunoassays such as ELISA or RIA. Such assays can be used to determine the dissociation constant of the antibody. The phrase “dissociation constant” refers to the affinity of an antibody for an antigen. Specificity of binding between an antibody and an antigen exists if the dissociation constant (K_{D}=1/K, where K is the affinity constant) of the antibody is, for example <1 μM, <100 nM, or <0.1 nM. Antibody molecules will typically have a K_{D} in the lower ranges. K_{D}=[Ab-Ag]/[Ab][Ag] where [Ab] is the concentration at equilibrium of the antibody, [Ag] is the concentration at equilibrium of the antigen and [Ab-Ag] is the concentration at equilibrium of the antibody-antigen complex. Typically, the binding interactions between antigen and antibody include reversible noncovalent associations such as electrostatic attraction, Van der Waals forces and hydrogen bonds.

D. Polypeptide Modulators

Modulators of the TGF-β or PI3K/Akt pathway can also be other types of compounds, such as polypeptides, including fusion proteins. In some embodiments herein, the TGF-β or TGF-β receptor inhibitor is a polypeptide, such as sTbRII:Fc. sTbRII:Fc is a soluble transmembrane domain of TGF-β receptor II fused to Fc. This fusion protein binds TGF-β₁ and TGF-β₃. In other examples the polypeptide inhibitor is betaglycan, which is also known as TGF-β receptor III. Betaglycan binds to various members of the TGF-β family of ligands. This molecule is not involved directly in TGF-β signal transduction, but acts as a reservoir for ligands of TGF-β receptors, thereby functioning as an inhibitor of the TGF-β pathway.

### VI. Methods of Treating Squamous Cell Carcinoma

As disclosed herein, the PI3K/Akt and TGF-β pathways act cooperatively to promote squamous cell carcinoma, such as HNSCC. Accordingly, provided herein is a method of treating a subject with SCC, by selecting a subject in need of treatment and treating that subject for SCC, for example by administering to the subject a therapeutically effective amount of an anti-cancer agent, such as an inhibitor of the PI3K/Akt pathway and a therapeutically effective amount of a modulator of the TGF-β pathway. Administration of the agent(s), such as the inhibitor and modulator, results in reduction in tumor size, inhibition of tumor growth, inhibition of tumor metastasis or inhibition of tumor progression, thereby treating the subject diagnosed with SCC.

Anti-cancer agents, such as modulators and inhibitors of the TGF-β and PI3K/Akt pathways are administered in any suitable manner, preferably with pharmaceutically acceptable carriers. Pharmaceutically acceptable carriers are determined in part by the particular composition being administered, as well as by the particular method used to administer the composition. Accordingly, there is a wide variety of suitable formulations of pharmaceutical compositions of the present disclosure.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Administration can be achieved using any suitable route. In some embodiments, administration is oral. In other embodiments, administration is topical. In other embodiments, administration is systemic.

In some embodiments, the inhibitor of the PI3K/Akt pathway and the modulator of the TGF-β pathway are administered simultaneously, such as part of a single composition, or as individual compositions that are co-administered. As used herein, “administered simultaneously” includes administration of two individual compositions that occurs up to about one hour apart. In other embodiments, the inhibitor of the PI3K/Akt pathway and the modulator of the TGF-β pathway are administered in succession. When administered separately, the inhibitor of the PI3K/Akt pathway can be administered first or the modulator of the TGF-β pathway can be administered first. The length of time between administration of the two compounds can vary, such as about two hours, about 4 hours, about 8 hours, about 12 hours, about 24 hours, about 2 days, about 3 days, about 5 days, about 7 days, about 10 days, about 14 days, or about 1 month.

Administration of the inhibitor of the PI3K/Akt pathway and the modulator of the TGF-β pathway (as a single composition or as two individual compositions) can be accomplished by single or multiple doses. The dose required will vary from subject to subject depending on the species, age, weight and general condition of the subject, the particular inhibitor or modulator being used and its mode of administration. An appropriate dose can be determined by one of ordinary skill in the art using only routine experimentation. If administered in multiple doses, the time between delivery of each dose can vary between days, weeks, months and years.

Administration of inhibitors and modulators of the PI3K/Akt and TGF-β pathways can also be accompanied by administration of other anti-cancer agents or therapeutic treatments (such as surgical resection of a tumor). Any suitable anti-cancer agent can be administered in combination with inhibitors and modulators of the PI3K/Akt and TGF-β pathways. Exemplary anti-cancer agents include, but are not limited to, chemotherapeutic agents, such as, for example, mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones (e.g. anti-androgens) and anti-angiogenesis agents. Other anti-cancer treatments include radiation therapy and antibodies that specifically target cancer cells.

Non-limiting examples of alkylating agents include nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine).

Non-limiting examples of antimetabolites include folic acid analogs (such as methotrexate), pyrimidine analogs (such as 5-FU or cytarabine), and purine analogs, such as mercaptopurine or thioguanine.

Non-limiting examples of natural products include vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitocycin C), and enzymes (such as L-asparaginase).

Non-limiting examples of miscellaneous agents include platinum coordination complexes (such as cis-diamine-dichloroplatinum II also known as cisplatin), substituted ureas (such as hydroxyurea), methyl hydrazine derivatives (such as procarbazine), and adrenocrotical suppressants (such as mitotane and aminoglutethimide).

Non-limiting examples of hormones and antagonists include adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testerone proprionate and fluoxymesterone). Examples of the most commonly used chemotherapy drugs include Adriamycin, Alkeran, Ara-C, BiCNU, Busulfan, CCNU, Carboplatinum, Cisplatinum, Cytoxan, Daunorubicin, DTIC, 5-FU, Fludarabine, Hydrea, Idarubicin, Ifosfamide, Methotrexate, Mithramycin, Mitomycin, Mitoxantrone, Nitrogen Mustard, Taxol (or other taxanes, such as docetaxel), Velban, Vincristine, VP-16, while some more newer drugs include Gemcitabine (Gemzar), Herceptin, Irinotecan (Camptosar, CPT-11), Leustatin, Navelbine, Rituxan STI-571, Taxotere, Topotecan (Hycamtin), Xeloda (Capecitabine), Zevelin and calcitriol.

Non-limiting examples of immunomodulators that can be used include AS-101 (Wyeth-Ayerst Labs), bropirimine (Upjohn), gamma interferon (Genentech), GM-CSF (granulocyte macrophage colony stimulating factor; Genetics Institute), IL-2 (Cetus or Hoffman-LaRoche), human immune globulin (Cutter Biological), IMREG (from Imreg of New Orleans, La.), SK&F 106528, and TNF (tumor necrosis factor; Genentech).

Common anti-cancer treatments for HNSCC include, but are not limited to, taxol (a chemotherapeutic agent); carboplatin (a chemotherapeutic agent); cisplatin (a chemotherapeutic agent); cetuximab (also known as Erbitux™; a monoclonal antibody against EGFR); bevacizumab (VEGF inhibitor, an anti-angiogenesis agent); and erlotinib (an EGFR inhibitor). Co-administration of an anti-cancer drug can occur concomitantly with administration of the inhibitors and modulators of the PI3K/Akt and TGF-β pathways, or the anti-cancer drug can be administered separately.

Another treatment of SCC is surgical treatment, for example surgical resection of the cancer or a portion of it. Another example of a treatment is radiotherapy, for example administration of radioactive material or energy (such as external beam therapy) to the tumor site to help eradicate the tumor or shrink it prior to surgical resection.

### VII. Methods for Diagnosis of Squamous Cell Carcinoma

Provided herein is a method of diagnosing a subject as having cancer, such as SCC, or being susceptible to developing cancer, by detecting the presence or absence of at least one tumor-associated mutation in the TGFBR1 gene and at least one tumor-associated mutation in the PTEN gene. The presence of the mutations indicates the subject has cancer, or has increased susceptibility to developing cancer. Further provided is a method of diagnosing a subject as having cancer, such as SCC, or being susceptible to developing cancer, by detecting expression of TGFBR1 and PTEN in a sample obtained from the subject. A decrease in expression of TGFBR1 and PTEN relative to a control indicates the subject has cancer, or has increased susceptibility to developing cancer.

Methods of detecting mutations in a gene or protein of interest, and methods of detecting an alteration in expression of a gene of interest (such as an alternation in mRNA or protein expression) are well known. Exemplary detection methods are described below, but are not intended to be limiting.

A. Mutations in TGFBR1 and PTEN

Mutations and polymorphisms of TGFBR1 have been described. For example, TGFBR1 (6A), a 9 bp deletion coding for 3 alanine residues within the 9 alanine repeat region of exon 1, has been associated with HNSCC (Chen et al., Int J Cancer 93:653-661, 2001; Knobloch et al., Mutat Res 479:131-139 2001; Pasch et al., JAMA 294:1634-1646, 2005). However, the precise molecular nature of TGFBR1-mediated pro-oncogenic effects is still unknown. A previous study showed that 35% of mice with a targeted deletion of Tgfbr1 developed spontaneous SCCs in periorbital and/or perianal regions (Honjo et al., Cell Cycle 6:1360-1366 2007). As described herein, it was found that 45% of mice with a targeted deletion of Tgfbr1 in head and neck epithelia and DMBA treatment developed SCCs in head and neck regions. The PI3K/Akt pathway is activated in SCCs that develop in Tgfbr1 cKO mice, suggesting the critical role of the TGF-β signaling pathway and its crosstalk with the PI3K/Akt pathway in suppressing head and neck carcinogenesis.

PI3K/Akt is an important driver of cell proliferation and cell survival. The tumor suppressor PTEN, which is located on chromosome 10, is a negative regulator of the PI3K signaling pathway. As described herein, when PTEN is deleted, mutated or otherwise inactivated, activation of the PI3K pathway occurs without any exogenous stimulus, resulting in initiation of tumorigenesis. In HNSCC, PTEN mutations have been identified in 23% of cancer patients. The missense mutations of PTEN occurred in exons 5, 6, 7, and 8. Loss of heterozygosity (LOH) of PTEN locus was identified in 71% of HNSCC (Poetsch et al., Cancer Genet Cytogenet. 132(1):20-24, 2002). Loss of PTEN protein expression was observed in 29% of tongue cancer (Lee et al., Arch Otolaryngol Head Neck Surg. 127(12):1441-1445, 2001) and 47% of HNSCC cases showed at least one of the molecular alterations in the PI3K/Akt pathways including PI3KCA and AKT2 amplification, p110α overexpression, and PTEN protein downregulation (Pedrero et al., Int J Cancer 114(2):242-248, 2005). Spontaneous SCCs have been found in a mouse model with targeted deletion of PTEN in the epidermis using Keratin-5 Cre (Suzuki et al., Cancer Res 63(3):674-681, 2003).

B. Detecting Mutations in TGFBR1 and PTEN

Detecting mutations in TGFBR1 or PTEN can be accomplished using any technique known in the art. For example, the presence or absence of a TGFBR1 or PTEN mutation can be determined by conventional methods such as gene or RNA detection methods (for example, DNA sequencing, oligonucleotide hybridization, polymerase chain reaction (PCR) amplification with primers specific to the mutation), or protein detection methods (for example, immunoassays or biochemical assays to identify a mutated TGFBR1 or PTEN protein). Generally, the nucleic acid sequence of the TGFBR1 or PTEN gene or RNA in a sample can be detected by any suitable method or technique of detecting gene sequence. Such methods include, but are not limited to, PCR, reverse transcriptase-PCR (RT-PCR), in situ PCR, in situ hybridization, Southern blot, Northern blot, sequence analysis, microarray analysis, or other DNA/RNA hybridization platforms.

Detection of point mutations, insertions or deletions in target nucleic acids can be accomplished by molecular cloning of the target nucleic acid molecules and sequencing the nucleic acid molecules using techniques well known in the art. Alternatively, amplification techniques such as PCR can be used to amplify target nucleic acid sequences directly from a genomic DNA preparation from a tumor tissue or cell sample. The nucleic acid sequence of the amplified molecules can then be determined to identify mutations. Design and selection of appropriate primers is well within the abilities of one of ordinary skill in the art.

The ligase chain reaction (Wu et al., Genomics 4:560-569, 1989) and allele-specific PCR (Ruano and Kidd, Nucleic Acids Res. 17:8392, 1989) can also be used to amplify target nucleic acid sequences. Amplification by allele-specific PCR uses primers that hybridize at their 3′ ends to a particular target nucleic acid mutation. If the particular mutation is not present, an amplification product is not observed. Amplification Refractory Mutation System can also be used to detect mutations in nucleic acid sequences (U.S. Pat. No. 5,595,890; Newton et al., Nucleic Acids Res. 17:2503-2516, 1989). Insertions and deletions of genes can also be detected by cloning, sequencing and amplification. In addition, restriction fragment length polymorphism probes for the gene or surrounding marker genes can be used to score alteration of an allele or an insertion in a polymorphic fragment. Single stranded conformation polymorphism analysis can also be used to detect base change variants of an allele (Orita et al., Proc. Natl. Acad. Sci. USA 86:2766-2770, 1989). Other known techniques for detecting insertions and deletions can also be used with the claimed methods.

Mismatch detection can be used to detect point mutations in a target nucleic acid molecule, such as TGFBR1 or PTEN. Mismatches are hybridized nucleic acid duplexes which are not 100% complementary. The lack of total complementarity can be due to deletions, insertions, inversions, substitutions or frameshift mutations. An example of a mismatch cleavage technique is the RNase protection method, which is described in detail in Winter et al. (Proc. Natl. Acad. Sci. USA 82:7575-7579, 1985) and Myers et al. (Science 230:1242-1246, 1985). For example, detection of mutations in TGFBR1 or PTEN can involve the use of a labeled riboprobe that is complementary to wild-type TGFBR1 or PTEN. The riboprobe and nucleic acid molecule to be tested (for example, obtained from a tumor sample) are annealed (hybridized) together and subsequently digested with the enzyme RNase A, which is able to detect mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full-length duplex RNA for the riboprobe and the mRNA or DNA. The riboprobe need not be the full length of the target nucleic acid mRNA or gene, but can a portion of the target nucleic acid, provided it encompasses the position suspected of being mutated. If the riboprobe comprises only a segment of the target nucleic acid mRNA or gene, it may be desirable to use a number of these probes to screen the whole target nucleic acid sequence for mismatches if desired.

In a similar manner, DNA probes can be used to detect mismatches, for example through enzymatic or chemical cleavage (Cotton et al., Proc. Natl. Acad. Sci. USA 85: 4397, 1988; Shenk et al., Proc. Natl. Acad. Sci. USA 72:989, 1975). Alternatively, mismatches can be detected by shifts in the electrophoretic mobility of mismatched duplexes relative to matched duplexes (Cariello, Human Genetics 42:726, 1988). With either riboprobes or DNA probes, the target nucleic acid mRNA or DNA which may contain a mutation can be amplified before hybridization. Changes in target nucleic acid DNA can also be detected using Southern hybridization, especially if the changes are gross rearrangements, such as deletions and insertions.

Amplified nucleic acid sequences can also be screened using allele-specific probes. These probes are nucleic acid oligomers, each of which contains a region of the target nucleic acid gene harboring a known mutation. For example, one oligomer may be about 30 nucleotides in length, corresponding to a portion of the target gene sequence. By use of a battery of such allele-specific probes, target nucleic acid amplification products can be screened to identify the presence of a previously identified mutation in the target gene. Hybridization of allele-specific probes with amplified target nucleic acid sequences can be performed, for example, on a nylon filter. Hybridization to a particular probe under stringent hybridization conditions indicates the presence of the same mutation in the tumor tissue as in the allele-specific probe.

Target-specific primers are useful for determination of the nucleotide sequence of a target nucleic acid molecule using nucleic acid amplification techniques such as the polymerase chain reaction. Pairs of single stranded DNA primers can be annealed to sequences within or surrounding the target nucleic acid sequence in order to prime amplification of the target sequence. Allele-specific primers can also be used. Such primers anneal only to particular mutant target sequence, and thus will only amplify a product in the presence of the mutant target sequence as a template. In order to facilitate subsequent cloning of amplified sequences, primers may have restriction enzyme site sequences appended to their ends. Such enzymes and sites are well known in the art. The primers themselves can be synthesized using techniques which are well known in the art. Generally, the primers can be made using oligonucleotide synthesizing machines which are commercially available.

Nucleic acid probes that hybridize with a TGFBR1 or PTEN nucleic acid molecule, such as a wild-type TGFBR1 or PTEN nucleic acid molecule or a mutant TGFBR1 or PTEN nucleic acid molecule, are useful for a number of purposes. They can be used in Southern hybridization to genomic DNA and in RNase protection assays for detecting point mutations. The probes can also be used to detect target nucleic acid amplification products. TGFBR1 or PTEN probes can also be used to detect mismatches with the wild type gene or mRNA using other techniques. Mismatches can be detected using either enzymes (e.g., S1 nuclease), chemicals (e.g., hydroxylamine or osmium tetroxide and piperidine), or changes in electrophoretic mobility of mismatched hybrids as compared to totally matched hybrids (Novack et al., Proc. Natl. Acad. Sci. USA 83:586, 1986).

Mutations in nucleic acid molecules can also be detected by screening for alteration of the corresponding protein. For example, monoclonal antibodies immunoreactive with a target gene product can be used to screen a tissue, for example an antibody that is known to bind to a particular mutated position of the gene product (protein). For example, a suitable antibody may be one that binds to a deleted exon or that binds to a conformational epitope comprising a deleted portion of the target protein. Lack of cognate antigen would indicate a mutation. Such immunological assays can be accomplished using any convenient format known in the art, such as Western blot, immunohistochemical assay and enzyme-linked immunosorbent assay (ELISA).

C. Detecting Altered Expression of TGFBR1 and PTEN mRNA and Protein

As described below, expression of TGFBR1 and PTEN can be detected using any one of a number of methods well known in the art. Expression of either mRNA or protein is contemplated herein.

i. Methods for Detection of mRNA

In some embodiments, RNA is isolated from a sample of a subject, such as a fluid sample or tissue sample. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995). In one example, RNA isolation can be performed using purification kit, buffer set and protease from commercial manufacturers, such as QIAGEN®, according to the manufacturer's instructions. For example, total RNA from cells in culture (such as those obtained from a subject) can be isolated using QIAGEN® RNeasy mini-columns. Other commercially available RNA isolation kits include MASTERPURE®. Complete DNA and RNA Purification Kit (EPICENTRE® Madison, Wis.), and Paraffin Block RNA Isolation Kit (Ambion, Inc.). Total RNA from tissue samples can be isolated using RNA Stat-60 (Tel-Test). RNA prepared from tumor or other biological sample can be isolated, for example, by cesium chloride density gradient centrifugation.

Methods of gene expression analysis include methods based on hybridization of polynucleotides, methods based on sequencing of polynucleotides, and proteomics-based methods. In some examples, mRNA expression in a sample is quantified using northern blotting or in situ hybridization (Parker & Barnes, Methods in Molecular Biology 106:247-283, 1999); RNAse protection assays (Hod, Biotechniques 13:852-4, 1992); and PCR-based methods, such as reverse transcription polymerase chain reaction (RT-PCR) (Weis et al., Trends in Genetics 8:263-4, 1992). Alternatively, antibodies can be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS). In one example, RT-PCR can be used to compare mRNA levels in different samples, in normal and tumor tissues, with or without drug treatment, to characterize patterns of gene expression, to discriminate between closely related mRNAs, and to analyze RNA structure.

Methods for quantitating mRNA are well known in the art. In one example, the method utilizes RT-PCR. Generally, the first step in gene expression profiling by RT-PCR is the reverse transcription of the RNA template into cDNA, followed by its exponential amplification in a PCR reaction. Two commonly used reverse transcriptases are avian myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN® RT-PCR can be performed using commercially available equipment, such as, for example, ABI PRISM 7700® Sequence Detection System® (Perkin-Elmer-Applied Biosystems, Foster City, Calif.), or Lightcycler (Roche Molecular Biochemicals, Mannheim, Germany). In one example, the 5′ nuclease procedure is run on a real-time quantitative PCR device such as the ABI PRISM 7700® Sequence Detection System®. The system includes of thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

To minimize errors and the effect of sample-to-sample variation, RT-PCR can be performed using an internal standard. The ideal internal standard is expressed at a constant level among different tissues, and is unaffected by the experimental treatment. RNAs commonly used to normalize patterns of gene expression are mRNAs for the housekeeping genes glyceraldehyde-3-phosphate-dehydrogenase (GAPDH), beta-actin, and 18S ribosomal RNA.

A variation of RT-PCR is real time quantitative RT-PCR, which measures PCR product accumulation through a dual-labeled fluorogenic probe (e.g. TAQMAN® probe). Real time PCR is compatible both with quantitative competitive PCR, where internal competitor for each target sequence is used for normalization, and with quantitative comparative PCR using a normalization gene contained within the sample, or a housekeeping gene for RT-PCR (see Held et al., Genome Research 6:986 994, 1996). Quantitative PCR is also described in U.S. Pat. No. 5,538,848. Related probes and quantitative amplification procedures are described in U.S. Pat. No. 5,716,784 and U.S. Pat. No. 5,723,591. Instruments for carrying out quantitative PCR in microtiter plates are available from PE Applied Biosystems, 850 Lincoln Centre Drive, Foster City, Calif. 94404 under the trademark ABI PRISM® 7700.

The steps of a representative protocol for quantitating gene expression using fixed, paraffin-embedded tissues as the RNA source, including mRNA isolation, purification, primer extension and amplification are given in various published journal articles (see Godfrey et al., J. Mol. Diag. 2:84-91, 2000; Specht et al., Am. J. Pathol. 158:419-429, 2001).

An alternative quantitative nucleic acid amplification procedure is described in U.S. Pat. No. 5,219,727. In this procedure, the amount of a target sequence in a sample is determined by simultaneously amplifying the target sequence and an internal standard nucleic acid segment. The amount of amplified DNA from each segment is determined and compared to a standard curve to determine the amount of the target nucleic acid segment that was present in the sample prior to amplification.

In some embodiments of this method, the expression of a “housekeeping” gene or “internal control” can also be evaluated. These terms include any constitutively or globally expressed gene whose presence enables an assessment of mRNA levels. Such an assessment includes a determination of the overall constitutive level of gene transcription and a control for variations in RNA recovery.

In some examples, gene expression is identified or confirmed using the microarray technique. Thus, the expression profile can be measured in either fresh or paraffin-embedded tissue or cells, using microarray technology. In this method, TGFBR1 and PTEN nucleic acid sequences of interest (including cDNAs and oligonucleotides) are plated, or arrayed, on a microchip substrate. The arrayed sequences are then hybridized with specific DNA probes from cells or tissues of interest.

Serial analysis of gene expression (SAGE) is another method that allows the simultaneous and quantitative analysis of a large number of gene transcripts, without the need of providing an individual hybridization probe for each transcript. First, a short sequence tag (about 10-14 base pairs) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag (see, for example, Velculescu et al., Science 270:484-487, 1995; and Velculescu et al., Cell 88:243-251, 1997).

In situ hybridization (ISH) is another method for detecting and comparing expression of genes of interest. ISH applies and extrapolates the technology of nucleic acid hybridization to the single cell level, and, in combination with the art of cytochemistry, immunocytochemistry and immunohistochemistry, permits the maintenance of morphology and the identification of cellular markers to be maintained and identified, and allows the localization of sequences to specific cells within populations, such as tissues and blood samples. ISH is a type of hybridization that uses a complementary nucleic acid to localize one or more specific nucleic acid sequences in a portion or section of tissue (in situ), or, if the tissue is small enough, in the entire tissue (whole mount ISH).

Sample cells or tissues are treated to increase their permeability to allow a probe to enter the cells. The probe is added to the treated cells, allowed to hybridize at pertinent temperature, and excess probe is washed away. A complementary probe is labeled with a radioactive, fluorescent or antigenic tag, so that the probe's location and quantity in the tissue can be determined using autoradiography, fluorescence microscopy or immunoassay. The sample may be any sample as herein described, such as a non-cancerous or colon adenocarcinoma sample. Since the sequences of the genes of interest are known, probes can be designed accordingly such that the probes specifically bind the gene of interest.

In situ PCR is the PCR based amplification of the target nucleic acid sequences prior to ISH. For detection of RNA, an intracellular reverse transcription step is introduced to generate complementary DNA from RNA templates prior to in situ PCR. This enables detection of low copy RNA sequences.

Prior to in situ PCR, cells or tissue samples are fixed and permeabilized to preserve morphology and permit access of the PCR reagents to the intracellular sequences to be amplified. PCR amplification of target sequences is next performed either in intact cells held in suspension or directly in cytocentrifuge preparations or tissue sections on glass slides. In the former approach, fixed cells suspended in the PCR reaction mixture are thermally cycled using conventional thermal cyclers. After PCR, the cells are cytocentrifuged onto glass slides with visualization of intracellular PCR products by ISH or immunohistochemistry. In situ PCR on glass slides is performed by overlaying the samples with the PCR mixture under a coverslip which is then sealed to prevent evaporation of the reaction mixture. Thermal cycling is achieved by placing the glass slides either directly on top of the heating block of a conventional or specially designed thermal cycler or by using thermal cycling ovens.

Detection of intracellular PCR products is generally achieved by one of two different techniques, indirect in situ PCR by ISH with PCR-product specific probes, or direct in situ PCR without ISH through direct detection of labeled nucleotides (such as digoxigenin-11-dUTP, fluorescein-dUTP, 3H-CTP or biotin-16-dUTP), which have been incorporated into the PCR products during thermal cycling.

iii. Methods for Detection of Protein

In some examples, expression of TGFBR1 and PTEN proteins is analyzed in a sample obtained from a subject, such as a blood sample or a tissue sample (such as epithelial cells from the head or neck region of the subject). A reduction in the amount of TGFBR1 and/or PTEN proteins in the sample relative to a control (such as a sample from a healthy subject or a standard value) allows for diagnosis of SCC in a subject.

The availability of antibodies specific to TGFBR1 and PTEN proteins facilitates the detection and quantitation of inflammatory proteins by one of a number of immunoassay methods that are well known in the art, such as those presented in Harlow and Lane (Antibodies, A Laboratory Manual, CSHL, New York, 1988). Methods of constructing such antibodies are known in the art. It should be noted that antibodies to TGFBR1 and PTEN are available from several commercial sources.

Any standard immunoassay format (such as ELISA, Western blot, or RIA assay) can be used to measure protein levels. Thus, TGFBR1 and PTEN polypeptide levels in a sample can readily be evaluated using these methods. Immunohistochemical techniques can also be utilized for TGFBR1 and PTEN protein detection and quantification. General guidance regarding such techniques can be found in Bancroft and Stevens (Theory and Practice of Histological Techniques, Churchill Livingstone, 1982) and Ausubel et al. (Current Protocols in Molecular Biology, John Wiley & Sons, New York, 1998).

For the purposes of quantitating TGFBR1 and PTEN proteins, a biological sample of the subject that includes cellular proteins can be used. Quantitation of TGFBR1 and PTEN protein can be achieved by immunoassay. The amount of TGFBR1 and PTEN protein can be assessed in a sample obtained a test subject, and in some cases, in a sample obtained from a healthy subject. A significant increase or decrease in the amount can be evaluated using statistical methods disclosed herein and/or known in the art.

Quantitative spectroscopic approaches methods, such as SELDI, can be used to analyze TGFBR1 and PTEN expression in a sample. In one example, surface-enhanced laser desorption-ionization time-of-flight (SELDI-TOF) mass spectrometry is used to detect protein expression, for example by using the ProteinChip™ (Ciphergen Biosystems, Palo Alto, Calif.). Such methods are well known in the art (for example see U.S. Pat. No. 5,719,060; U.S. Pat. No. 6,897,072; and U.S. Pat. No. 6,881,586). SELDI is a solid phase method for desorption in which the analyte is presented to the energy stream on a surface that enhances analyte capture or desorption.

Briefly, one version of SELDI uses a chromatographic surface with a chemistry that selectively captures analytes of interest, such as inflammatory proteins. Chromatographic surfaces can be composed of hydrophobic, hydrophilic, ion exchange, immobilized metal, or other chemistries. For example, the surface chemistry can include binding functionalities based on oxygen-dependent, carbon-dependent, sulfur-dependent, and/or nitrogen-dependent means of covalent or noncovalent immobilization of analytes. The activated surfaces are used to covalently immobilize specific “bait” molecules such as antibodies, receptors, or oligonucleotides often used for biomolecular interaction studies such as protein-protein and protein-DNA interactions.

The surface chemistry allows the bound analytes to be retained and unbound materials to be washed away. Subsequently, analytes bound to the surface (such as inflammatory proteins) can be desorbed and analyzed by any of several means, for example using mass spectrometry. When the analyte is ionized in the process of desorption, such as in laser desorption/ionization mass spectrometry, the detector can be an ion detector. Mass spectrometers generally include means for determining the time-of-flight of desorbed ions. This information is converted to mass. However, one need not determine the mass of desorbed ions to resolve and detect them: the fact that ionized analytes strike the detector at different times provides detection and resolution of them. Alternatively, the analyte can be detectably labeled (for example with a fluorophore or radioactive isotope). In these cases, the detector can be a fluorescence or radioactivity detector. A plurality of detection means can be implemented in series to fully interrogate the analyte components and function associated with retained molecules at each location in the array.

In another example, antibodies are immobilized onto the surface using a bacterial Fc binding support. The chromatographic surface is incubated with a sample, and the antigens present in the sample can recognize the antibodies on the chromatographic surface. The unbound proteins and mass spectrometric interfering compounds are washed away and the proteins that are retained on the chromatographic surface are analyzed and detected by SELDI-TOF. The MS profile from the sample can be then compared using differential protein expression mapping, whereby relative expression levels of proteins at specific molecular weights are compared by a variety of statistical techniques and bioinformatic software systems.

D. Output Devices for Diagnostic Results

Mutations in a gene or encoded protein and/or gene expression can be evaluated using any technique described above, or any other method known in the art. As described herein, gene expression can be measured, for example, using labeled probes that can be detected using standard equipment. For example, gene expression measurements using microarray or RT-PCR (which typically use labeled probes specific for a gene product) can be quantitated using a microarray scanner or other suitable scanner for detecting the label. In addition, mutations in a gene or corresponding mRNA can be detected by direct sequencing of a nucleic acid molecule, detection of an amplification product, microarray analysis or any other DNA/RNA hybridization platform. For detection of mutant proteins, an immunoassay, biochemical assay or microarray can be used.

The diagnostic results of gene expression and mutation analyses can be transmitted using any one of a number of output devices or formats known in the art. For example, the output device can be a visual output device, such as a computer screen or a printed piece of paper. In other examples, the output device can be an auditory output device, such as a speaker. In other examples, the output device is a printer. In some cases, the diagnostic results are recorded in a patient's printed or electronic medical record.

E. Other Diagnostic Methods

In some embodiments of the diagnostic methods disclosed herein, if the diagnostic test indicates the subject has SCC, or is susceptible to developing SCC, the subject is subjected to additional diagnostic tests to confirm the diagnosis by other means. Alternatively, the test is used to confirm a diagnosis already indicated by other means. Any one of a number of means known in the art of diagnosing a subject with cancer, such as SCC, can be used. Other means of diagnosing SCC, or confirming a diagnosis of SCC, can include diagnostic modalities such as physical examination, clinical suspicion, tissue biopsy, analysis of additional mutations associated with SCC or a specific sub-type of SCC (such as HNSCC), or histological examination, for example tissue biopsy with histological diagnosis by a pathologist. In some cases, a patient undergoes a physical examination to identify any suspicious lesions (such as a tumor). If a suspicious lesion is identified, typically a biopsy is taken, which can be used to identify tumor-associated mutations (such as tumor-associated mutations in TGFBR1 and PTEN, or other genes that play a role in the development of progression of cancer), to detect expression levels of TGFBR1 and PTEN (or expression of other genes known to play a role in the development of progression of cancer), and/or to histologically examine the tissue to detect malignant cells.

### VIII. Genetically Modified Animals and Uses Thereof

Provided herein is a genetically modified non-human animal comprising a homozygous deletion of the TGFBR1 gene and a homozygous deletion of the PTEN gene. As disclosed herein, such genetically modified animals are highly susceptible to developing SCC tumors, such as HNSCC tumors. In some embodiments, the genetically modified non-human animal is a rodent, such as a mouse. In some embodiments, the deletion of the TGFBR1 gene and the deletion of the PTEN gene are conditional deletions. In some embodiments, the deletions occur only in the head and neck epithelia of the animal. In a particular example disclosed herein, conditional deletion of TGFBR1 and PTEN occur following exposure of a genetically modified mouse to tamoxifen, which drives expression of Cre recombinase, resulting in conditional deletion of TGFBR1 and PTEN. Exemplary methods of generating genetically modified animals is well known in the art and are described below.

A method of screening therapeutic agents useful for the treatment of cancer, such as SCC, is also provided herein. The screening method comprises (i) providing a genetically modified non-human animal with a homozygous deletion of the TGFBR1 gene and a homozygous deletion of the PTEN gene; (ii) administering a candidate therapeutic agent to the genetically modified animal; and (iii) determining the effect of administering the candidate therapeutic agent to the genetically modified animal. A reduction in tumor size, inhibition of tumor growth, inhibition of tumor metastasis or inhibition of tumor progression in the genetically modified animal identifies the candidate agent as a therapeutic agent useful for the treatment of cancer. Candidate therapeutic agents can be any type of compound, such as an antibody, polypeptide, polynucleotide, small molecule or antisense compound.

Genetically modified animals are also referred to herein as “transgenic animals.” Any transgenic animal can be of use in the methods disclosed herein, provided the transgenic animal is a non-human animal. A “non-human animal” includes, but is not limited to, a non-human primate, a farm animal such as swine, cattle, and poultry, a sport animal or pet such as dogs, cats, horses, hamsters, rodents, or a zoo animal such as lions, tigers or bears. In one specific, non-limiting example, the non-human animal is a transgenic animal, such as, but not limited to, a transgenic mouse, cow, sheep, or goat. In one specific, non-limiting example, the transgenic animal is a mouse. In a particular example, the transgenic animal has altered proliferation and/or differentiation of a cell type as compared to a non-transgenic control (wild-type) animal of the same species.

A transgenic animal contains cells that bear genetic information received, directly or indirectly, by deliberate genetic manipulation at the subcellular level, such as by microinjection or infection with a recombinant virus, such that a recombinant DNA is included in the cells of the animal. This molecule can be integrated within the animal's chromosomes, or can be included as extrachromosomally replicating DNA sequences, such as might be engineered into yeast artificial chromosomes. A transgenic animal can be a “germ cell line” transgenic animal, such that the genetic information has been taken up and incorporated into a germ line cell, therefore conferring the ability to transfer the information to offspring. If such offspring in fact possess some or all of that information, then they, too, are transgenic animals.

Transgenic animals can readily be produced by one of skill in the art. For example, transgenic animals can be produced by introducing into single cell embryos DNA encoding a marker, in a manner such that the polynucleotides are stably integrated into the DNA of germ line cells of the mature animal and inherited in normal Mendelian fashion. Advances in technologies for embryo micromanipulation permit introduction of heterologous DNA into fertilized mammalian ova. For instance, totipotent or pluripotent stem cells can be transformed by microinjection, calcium phosphate mediated precipitation, liposome fusion, retroviral infection or other means. The transformed cells are then introduced into the embryo, and the embryo then develops into a transgenic animal. In one non-limiting method, developing embryos are infected with a retrovirus containing the desired DNA, and a transgenic animal is produced from the infected embryo.

In another specific, non-limiting example, the appropriate DNA(s) are injected into the pronucleus or cytoplasm of embryos, preferably at the single cell stage, and the embryos are allowed to develop into mature transgenic animals. These techniques are well known. For instance, reviews of standard laboratory procedures for microinjection of heterologous DNAs into mammalian (mouse, pig, rabbit, sheep, goat, cow) fertilized ova include: Hogan et al., Manipulating the Mouse Embryo, Cold Spring Harbor Press, 1986; Krimpenfort et al., Bio/Technology 9:86, 1991; Palmiter et al., Cell 41:343, 1985; Kraemer et al., Genetic Manipulation of the Early Mammalian Embryo, Cold Spring Harbor Laboratory Press, 1985; Hammer et al., Nature 315:680, 1985; Purcel et al., Science 244:1281, 1986; U.S. Pat. No. 5,175,385; U.S. Pat. No. 5,175,384.

In addition, an exemplary method of producing a conditional knockout animal having homozygous deletions of TGFBR1 and PTEN is described in Example 1 below.

The following examples are provided to illustrate certain particular features and/or embodiments. These examples should not be construed to limit the disclosure to the particular features or embodiments described.

### EXAMPLES

### Example 1

### Materials and Methods

### Generation of Tgfbr1 cKO Mice

Tgfbr1^{f/f} mice (mixed genetic strains of C57BL/6, 129SV/J and FVB/N) (Larsson et al., EMBO J20:1663-73, 2001; Honjo et al., Cell Cycle 6:1360-6, 2007) were crossed with the K14-CreER^{tam} mouse line (genetic strain CD-1) (Vasioukhin et al., Proc Natl Acad Sci USA 96:8551-6, 1999) to generate mice heterozygous for both Tgfbr1 flox and K14-CreER^{tam} (K14-CreER^{tam}; Tgfbr1^{f/+}). The Tgfbr1 cKO mice (K14-CreER^{tam};Tgfbr1^{f/f}) were generated from crosses between mice heterozygous for both Tgfbr1 flox and K14-CreER^{tam} (K14-reER^{tam}; Tgfbr1^{f/+}) and mice homozygous for the Tgfbr1 flox allele (Tgfbr1^{f/f}). This breeding strategy resulted in the generation of Tgfbr1 cKO mice as well as Tgfbr1^{f/f}, Tgfbr1^{f/+}, and K14-CreER^{tam}; Tgfbr1^{f/+} mice. The Tgfbr1 cKO mice and their controls are from the same litter and therefore have exactly the same mixed genetic background. Mice were housed under a 12-hour light/dark cycle.

Littermates were genotyped at 1 week of age and grouped based on genotypes for the experiments. Tamoxifen (200 μl of 10 μg/μl in corn oil) was applied by gavage in the oral cavity of 3-month old Tgfbr1 cKO mice for 5 consecutive days to induce homozygous deletion of Tgfbr1 in head and neck epithelia. For tumor initiation with DMBA, a single dose of 50 μg DMBA (Sigma; dissolved in 100 μl corn oil) was applied orally to each group of mice 10 days after the last tamoxifen treatment. Paired K14-CreER^{tam}; Tgfbr1^{f/+} and Tgfbr1^{f/f} mice were also treated with the same dosage of tamoxifen and DMBA as controls. To study early premalignant lesions, mice from each group were dissected at 4 weeks after DMBA initiation. Once tumors developed in the oral cavity, mice were switched to soft food and monitored daily. Tumor-bearing mice were euthanized when tumor diameter approached 1 cm in size or if tumors were ulcerated and bleeding, or there was any sign of the mice suffering pain or weight loss resulting from tumors. Necropsy was performed on each euthanized mouse. Histological slides were prepared to identify primary tumors and metastases in the cervical lymph nodes, lungs, and brain. Head and neck tissues, including the buccal mucosa and tongue as well as other tissues like ear, esophagus, and forestomach, were also dissected.

### Histology, Immunostaining, and BrdU Labeling

All tissues were fixed overnight in buffered 4% paraformaldehyde, transferred to 70% ethanol, and embedded in paraffin. Five-micron sections were cut and stained with hematoxylin and eosin. For immunohistochemical studies, the following antibodies were used: TGFBR1 antibody (ab31013), CDKN1A (p21) antibody (ab7960), c-Myc antibody (ab32) (abcam, Cambridge, Mass.) at 1:200 dilution; phospho-Smad2 (Ser465/467) antibody (Millipore, Billerica, Mass.) at 1:500 dilution; mouse Ki-67 (TEC-3) antibody (DAKO, Carpinteria, Calif.) at 1:100 dilution; Cox-2 mouse monoclonal antibody (BD Transduction Laboratories, San Jose, Calif.) at 1:50; phospho-Akt (Ser473) mAb and phospho-mTOR (Ser2448) antibody (Cell Signaling Technology, Danvers, Mass.) at 1:100 dilution.

The tissue slides were dewaxed in xylenes, hydrated through graded alcohols, and incubated in 3% hydrogen peroxide in phosphate-buffered saline (PBS) for 30 minutes to block the endogenous peroxidase. After washing in distilled water, antigen retrieval was performed with 10 mM citric acid in a microwave for 20 minutes (2 minutes at 100% power and 18 minutes at 20% power). Slides were allowed to cool to room temperature, rinsed thoroughly with distilled water and PBS, then incubated in blocking solution (2.5% BSA in PBS) for 30 minutes at room temperature. Excess solution was discarded, and the sections were incubated overnight at 4° C. with the primary antibody diluted in blocking solution. After washing with PBS, the slides were sequentially incubated with the biotinylated secondary antibody (1:400; Vector, Burlingame, Calif.) for 30 minutes, followed by the avidin-biotin complex method (Vector Stain Elite, PK-6100 Standard ABC kit; Vector, Burlingame, Calif.) for 30 minutes at room temperature. The slides were washed and developed in 3′3′-diaminobenzidine (FASTDAB tablet; Sigma, St. Louis, Mo.) under microscopic control. The reaction was stopped in tap water, and the tissues were counterstained with hematoxylin, dehydrated, and mounted.

For BrdU labeling, mice were injected i.p. with 50 mg/kg body weight of BrdU (Sigma, St. Louis, Mo.) in sterile 1×PBS 4 hours before biopsy. BrdU immunostaining was performed on paraformaldehyde-fixed tissue sections using rat anti-BrdU antibody (Accurate Chemical & Scientific Corp., Westbury, N.Y.). For immunofluorescent staining, after incubation with primary antibody, the slides were incubated with fluorophore-conjugated secondary antibodies with 4′,6′-diamidino-2-phenylidole (DAPI) (Jackson ImmunoResearch Laboratories, Inc., West Grove, Pa.) for 1 hour in the dark at room temperature. The primary antibodies included the following: Keratin K₁₄ (Covance, Emeryville, Calif.), α-smooth muscle actin (ASM-1) antibody (Millipore, Billerica, Mass.), endoglin (CD105) antibody and TGF-β1 antibody (R&D Systems, Minneapolis, Minn.). Sodium borohydride and Sudan Black B (Sigma, St. Louis, Mo.) were used to reduce aldehyde and lipofuscin-induced fluorescence. Confocal microscopy images were obtained using a Zeiss LSM 510 NLO META confocal microscope (Zeiss, Thornwood, N.Y.).

Terminal Deoxyribonucleotidyl Transferase-Mediated dUTP Nick End Labeling Assay

Terminal deoxyribonucleotidyl transferase-mediated dUTP nick end labeling (TUNEL) assay was performed on paraformaldehyde-fixed tissue sections using the In situ Apoptosis Detection Kit and following the directions of the manufacturer (TaKaRa, Shiga, Japan).

### Assessment of Cre-Mediated Recombination

The Tgfbr1 cKO mice and controls (Tgfbr1^{f/f}) were dissected 10 days after tamoxifen treatment. Genomic DNA was extracted from the indicated tissues using DNeasy™ Blood & Tissue Kit (QIAGEN, Valencia, Calif.). Cre-mediated recombination of Tgfbr1^{f/f} allele was assessed using a PCR-based assay that only generated an amplicon if the Tgfbr1^{f/f} allele had undergone Cre-mediated recombination (Larsson et al., EMBO J. 20:1663-73, 2001).

### Flow Cytometry Analysis

Flow cytometry staining was performed as described before (Liu et al., Nat Immunol 9:632-40, 2008). Briefly, lymphocytes were isolated and stained with the indicated antibodies for the surface markers and subjected to flow cytometry analysis.

### Quantitative Real-Time PCR Analysis

Total RNA was isolated from buccal mucosa, tongue, and tumors of the Tgfbr1 cKO mice and controls (Tgfbr1^{f/f}) by using Trizol™ and chloroform. To determine the Tgfbr1 mRNA expression levels in head and neck epithelia, 1 μg of total RNA was used for RT-PCR analysis as described (Honjo et al., Cell Cycle 6:1360-6, 2007). The quantitative real-time PCR (qRT-PCR) was done in triplicate using samples from 5 mice.

### Western Blot Analysis

Normal buccal mucosa and tongue together with tumors that developed in DMBA-initiated Tgfbr1 cKO mice were carefully dissected from 6 pairs of Tgfbr1^{f/f} and Tgfbr1 cKO mice. Proteins were extracted from tissues using T-PER reagent (Pierce, Rockford, Ill.) with a complete mini protease inhibitor cocktail (Roche, Branchburg, N.J.). NuPAGE 4-12% Bis-Tris precast gel was used for electrophoresis on the XCell surelock Mini-Cell (Invitrogen, Carlsbad, Calif.). A total amount of 40 μg protein from each sample was denatured and then loaded in each lane. Proteins were then transferred on to a PVDF membrane. The following antibodies were used: TGFBR1 antibody (sc-398), CDKN1A (p21) antibody (sc-397), c-Myc antibody (sc-40) (Santa Cruz Biotechnology, Santa Cruz, Calif.) at 1:200 dilution; Smad2 antibody (Zymed, San Francisco, Calif.), phospho-Smad2 (Ser465/467) antibody (Millipore, Billerica, Mass.), PTEN antibody, and phosphor-PTEN (S380) antibody (R&D Systems, Inc. Minneapolis, Minn.) at 1:500 dilution; Akt antibody, phospho-Akt (Ser473) antibody (Cell Signaling Technology, Danvers, Mass.) at 1:100 dilution; and β-Actin antibody (Millipore, Billerica, Mass.) at 1:2000. The signals were visualized using a horseradish peroxidase-conjugated secondary antibody (Santa Cruz Biotechnology, Inc., Santa Cruz, Calif.) followed by chemiluminescence detection (Pierce, Rockford, Ill.). Subsequently, all blots were reincubated with anti β-Actin antibody and developed similarly as a loading controls.

### Statistical Analysis

Statistical differences in the levels of mRNA expression between controls and experimental samples were established using the Student's t-test.

### Example 2

### Inducible Deletion of Tgfbr1 in Head and Neck Epithelia is not Sufficient for SCCs Formation in Mice

To study the role of TGFBR1-mediated TGF-β signaling in the development of SCC, an inducible head- and neck-specific knockout mouse model was generated by crossing Tgfbr1 floxed mice with K14-CreER^{tam} mice. K14 is expressed in proliferating keratinocytes in the basal layer of the epidermis. The inducible features of Cre were achieved by fusing Cre recombinase to the tamoxifen responsive hormone-binding domain of the estrogen receptor (ER), which fails to bind estrogen but can be activated by an estrogen antagonist, tamoxifen (TM) (Vasioukhin et al., Proc Natl Acad Sci USA 96:8551-6, 1999). The expression of Cre is targeted by the human keratin 14 (K14) promoter. Upon applying TM (2 mg per mouse per day for 5 consecutive days) to the mouse oral cavity, CreER translocates from the cytoplasm to the nucleus, where it mediates the excision of Tgfbr1 exon3, resulting in deletion of Tgfbr1 in the mouse head and neck epithelia. Since the K14 promoter is also active in stem cells that regenerate the epidermis, sebaceous glands, hair follicles, and the oral mucosa, TM treatment causes permanent excision of Tgfbr1 in both epithelia and epidermis in the head and neck region including buccal mucosa, tongue as well as ears.

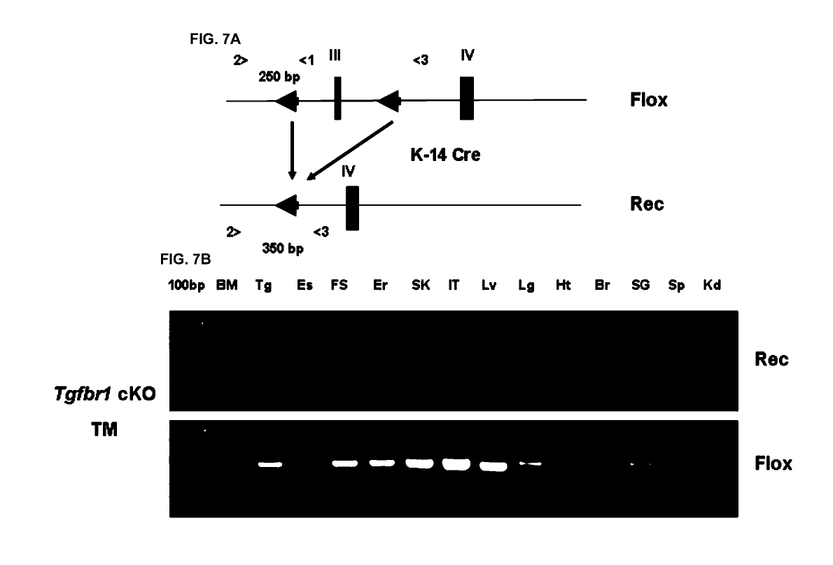

The Tgfbr1 cKO mice and controls (Tgfbr1^{f/f}) were dissected 10 days after TM treatment because the rate of renewal of the mouse stratified epithelia from the stem cells is about 1 week, which coincides with the mouse recovery period from TM toxicity. Genomic DNA was extracted from all major organs and tissues. Tissues from the head and neck area include the buccal mucosa, tongue, ear, esophagus, and forestomach. Cre-mediated recombination of the Tgfbr1^{f/f} allele was assessed using a PCR-based assay (FIG. 7A). Deletions of Tgfbr1 were detected in the buccal mucosa, tongue, and ear but not in the esophagus, forestomach, back skin, or any other nonstratified epithelial organs, such as the heart, lungs, liver, intestines, spleen, kidneys, or brain of Tgfbr1 cKO mice (FIG. 7B). No recombination was detected prior to TM administration.

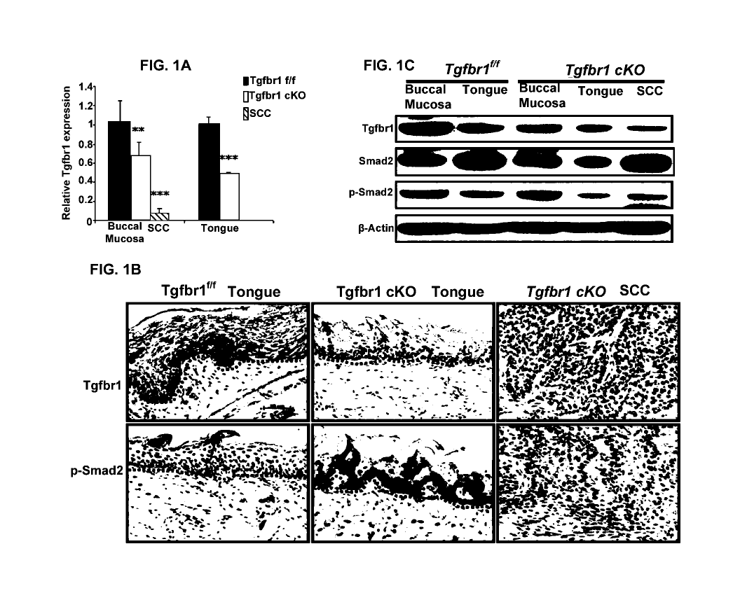

Tgfbr1 mRNA expression was examined by quantitative RT-PCR (qRT-PCR). The expression levels of Tgfbr1 mRNA in Tgfbr1^{f/f} mice were normalized as 1.00±0.23 in the buccal mucosa and 1.00±0.08 in the tongue. The mRNA expression levels were significantly reduced to a mean of 0.65±0.17 in the buccal mucosa (p<0.01) and 0.07±0.05 in SCC of Tgfbr1 cKO mice as well as 0.46±0.05 in the tongue (p<0.001) (FIG. 1A). Using immunostaining, the protein expression of Tgfbr1 was found to be significantly decreased in the buccal mucosa and in the tongue of Tgfbr1 cKO mice in comparison with those of Tgfbr1^{f/f} mice. A similar expression pattern was also observed when using antibody against phosphorylated Smad2, an activated mediator of TGF-β signaling (FIG. 1B). However, the expression of both Tgfbr1 and p-Smad2 in the skin epidermis and hair follicles of the same mice remained normal, suggesting that upon oral administration of TM, the deletion of Tgfbr1 and the inactivation of its downstream signaling was localized only in the head and neck epithelia. These results were further confirmed by Western blot (FIG. 1C).

Out of 31 Tgfbr1 cKO mice, only 3 (9.7%, 3/31) developed spontaneous tumors including two SCCs in the periorbital region and one in the upper lateral neck. No significant pathological changes in the head and neck region were observed in the remaining Tgfbr1 cKO mice during 1 year of observation. Thus, these results indicate that inactivation of TGF-β signaling alone is not sufficient to promote tumor formation in head and neck epithelia of these mice.

### Example 3

### Deletion of Tgfbr1 in the Head and Neck Epithelia Together with DMBA Initiation Induces SCCs in Mice

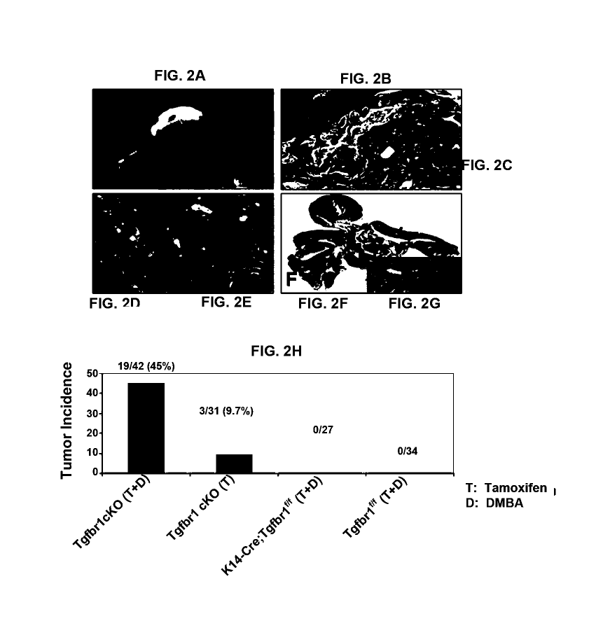

Because spontaneous tumor formation in Tgfbr1 cKO mice was rare, tumors in Tgfbr1 cKO mice were induced by applying a single dose (50 μg per mouse) of DMBA to the mouse oral cavity 10 days after the last TM treatment. DMBA is a commonly used chemical carcinogen for studying skin carcinogenesis. It can induce H-ras mutations that serve as a common initiating genetic event in sporadic cells (Kim et al., Anticancer Res 22:2733-40, 2002). After tumor initiation with DMBA, Tgfbr1 cKO mice started to develop SCCs in the head and neck area as early as 16 weeks, and by 1 year after treatment, 19 out of 42 (45%) Tgfbr1 cKO mice had developed SCCs (FIGS. 2B-2E). The sites of tumors that developed in DMBA-treated Tgfbr1 cKO mice included the oral cavity, periorbital region, muzzle area, and skin around the head and neck area (FIG. 2A). Approximately 16% ( 3/19) of mice with tumors developed metastases in the jugular lymph nodes and/or lungs by the time the mice were dissected (10-12 months after TM and DMBA treatment) (FIGS. 2F and 2G). Examination of H-ras mutations in 17 tumors found 9 (53%, 9/17) of them had A to T substitutions at codon 61 in exon 2 of the gene. K-ras mutations were also screened, but no mutations were detected in any of those tumors. No tumors developed in the heterozygous (K14-CreER^{tam};Tgfbr1^{f/+}, n=27) mice or the Tgfbr1 floxed homozygous (Tgfbr1^{f/f}, n=34) control littermates (also treated with TM and DMBA) during the same time period (FIG. 2H). However, only partial excision of Tgfbr1 in mouse head and neck epithelia were noted by IHC and Western blot due to the tamoxifen-induced K14Cre mouse line being used in this study (FIG. 1B, 1C). These results demonstrate that deletion of Tgfbr1 in mouse head and neck epithelia in combination with DMBA treatment results in development of SCCs.

### Example 4

### Enhanced Cell Proliferation, Inhibition of Apoptosis, and Down-Regulation of Cell Cycle 9 Inhibitors in the Head and Neck Epithelia of Tgfbr1 cKO Mice

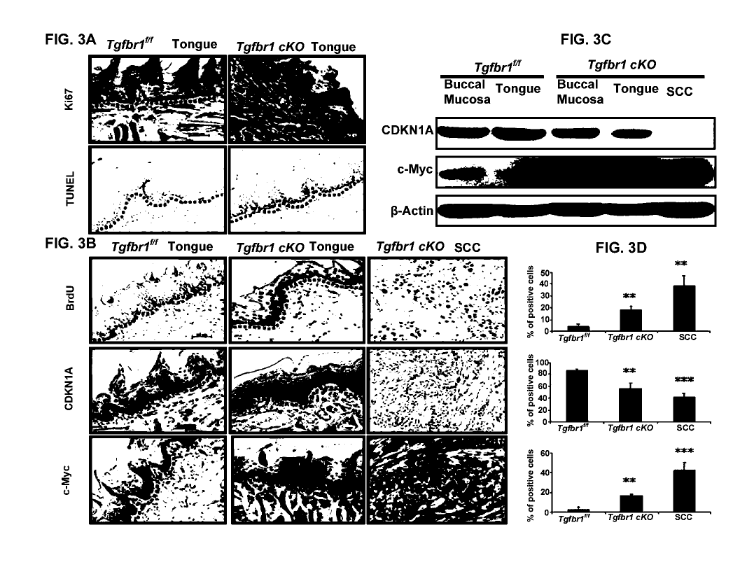

TGF-β has effects on both cell growth and apoptosis. Four weeks after DMBA treatment, an increased expression of a proliferative marker Ki67 was detected in the basal layer of the tongue of Tgfbr1 cKO mice but not in Tgfbr1^{f/f} mice. A decreased apoptosis was also observed, indicating that the imbalance between cell proliferation and apoptosis occurs early in the head and neck epithelia of Tgfbr1 cKO mice (FIG. 3A). Using BrdU assays, a significantly increased number of proliferative cells were found in Tgfbr1 cKO mice head and neck epithelia and SCCs when compared to those of Tgfbr1^{f/f} mice (FIGS. 3B and 3D). However, no apoptotic cells were observed in SCCs by TUNEL assay. Immunostaining revealed that CDKN1A expression was reduced in tongue and SCCs of Tgfbr1 cKO mice compared to that in Tgfbr1^{f/f} mice. In contrast, c-Myc was overexpressed in tongue of Tgfbr1 cKO mice and its expression was even more remarkable in SCCs (FIGS. 3B and 3D). These results were further confirmed by Western blot analysis (FIG. 3C). These results indicate the existence of an imbalance between cell proliferation, differentiation, and apoptosis in SCCs that developed in Tgfbr1 cKO mice, as well as in normal Tgfbr1 cKO mice head and neck epithelia.

### Example 5

### Enhanced TGF-β1 Paracrine Effect in Tumor Stroma of Tgfbr1 cKO Mice

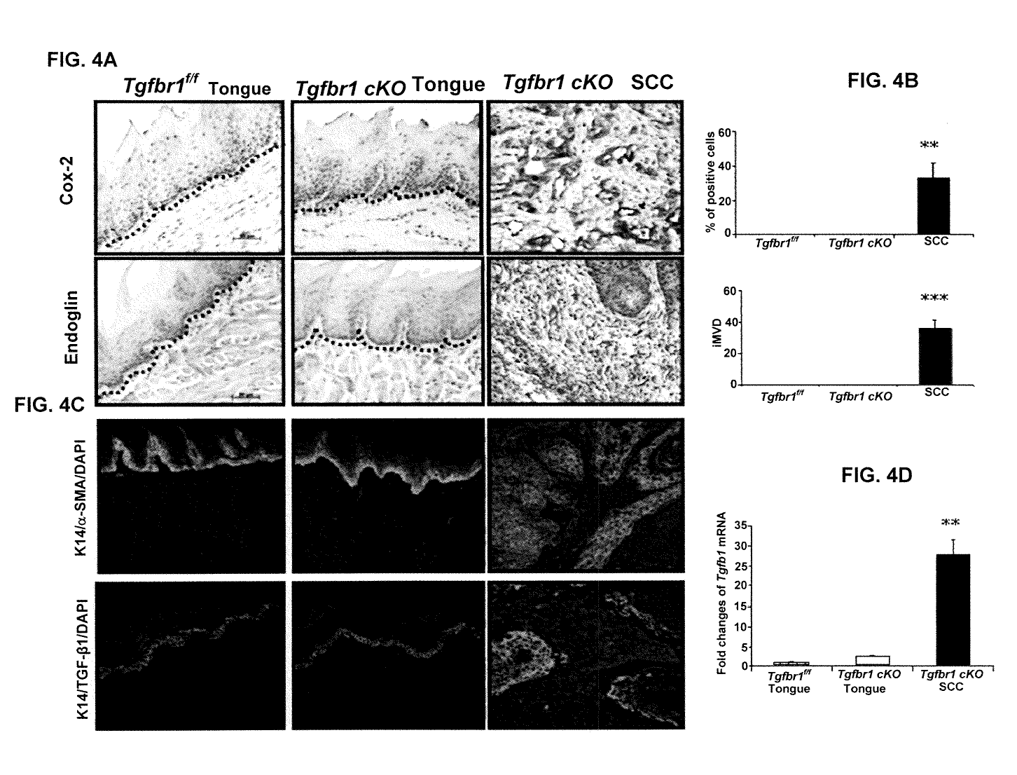

Increased inflammation and angiogenesis have been found in human HNSCCs (Chen et al., Clin Cancer Res 5:1369-79, 1999). Deletion of Tgfbr2 in mouse head and neck epithelia resulted in elevated endogenous TGF-β1 and enhanced the paracrine effect of TGF-β on tumor stroma (Lu et al., Genes Dev 20:1331-42, 2006). To investigate the paracrine effect of TGF-β in tumor progression in SCCs arising in the DMBA-treated Tgfbr1 cKO mice, the expression level of cyclooxygenase-2 (Cox-2) (Goulart et al., Am J Otolaryngol 30:89-94, 2009), endoglin (CD105) (Wikström et al., Prostate 51:268-75, 2002), and α-smooth muscle actin (SMA) was examined in tumor stromata (Orimo et al., Cell 121:335-48, 2005; Lewis et al., Br J Cancer 90:822-32, 2004). It was found that Cox-2 expression was absent in normal buccal mucosa and tongue of Tgfbr1^{f/f} mice as well as Tgfbr1 cKO mice, but its expression was significantly increased in SCCs, suggesting increased inflammation in tumors (FIGS. 4A and 4B). Using immunofluorescent staining, increased angiogenesis indicated by endoglin (CD105)-stained microvessels in the stromata surrounding SCCs were also observed (FIGS. 4A and 4B). It was also found that α-SMA, a hallmark of the myofibroblastic phenotype, was strongly expressed in the stroma surrounding SCCs but was not detected in the stroma of Tgfbr1^{f/f} mouse tongues (FIG. 4A). To determine whether increased inflammation, angiogenesis, and the myofibroblastic phenotype correlate with endogenous TGF-β1 levels in the area surrounding the SCCs, TGF-β1 expression was examined by qRT-PCR. In comparison with tissues from Tgfbr1^{f/f} mice, the levels of TGF-β1 expression were increased 2.42±0.31 fold and 27.08±4.42 fold (p<0.01) in DMBA-treated Tgfbr1 cKO mice tongues and SCCs, respectively (FIG. 4D). Immunofluorescent staining indicated significantly increased expression of TGF-β1 only in the tumor stroma (FIG. 4C).

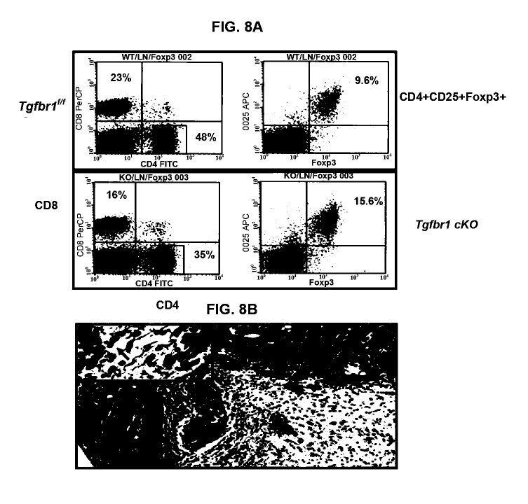

Evasion of the immune response is one of the most important features of TGF-β-mediated tumor progression (Smyth et al., Adv Immunol 90:1-50, 2006; Kim et al., Nature 441:1015-9, 2006). In order to understand the molecular mechanisms that underlie tumor formation in this mouse model, especially the role that evasion of the immune response plays in this tumorigenesis, the immune status of the Tgfbr1 cKO mice was examined using flow cytometry analysis. Compared with their WT control littermates, Tgfbr1 cKO mice showed significantly reduced numbers of both CD4+ and CD8+ effector T cells, whereas the regulatory T cells CD4+ CD25+Foxp3+ were increased, indicating active immune suppression in Tgfbr1 cKO mice (FIG. 8A). Gross changes in inflammation within tumors were noted by H&E staining (FIG. 8B).

### Example 6

### Activation of PI3K/Akt Signaling in SCCs of Tgfbr1 cKO Mice

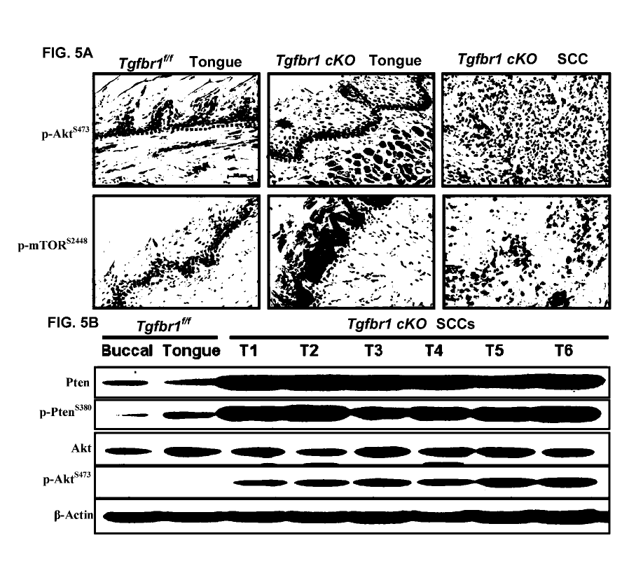

The PI3K/Akt pathway is important in suppressing apoptosis and in promoting cell growth and proliferation. In cancer cells, this pathway can be deregulated in multiple ways. For example, in HNSCC hyperactivation of PI3K can be induced by mutations or by enhanced activity of its upstream activators, including the Ras oncoproteins or inactivation of PTEN (phosphatase and tensin homolog deleted on chromosome 10) (Molinolo et al., Oral Oncol 45(4-5):324-334, 2008). PTEN is a potent tumor suppressor gene and a negative regulator of the PI3K/Akt pathway. Mutations of PTEN have been found in a wide range of human cancers (Eng, Hum Mutat 22:183-98, 2003). In this study, a significantly increased level of unphosphorylated PTEN was detected in all of the tumors that developed in the DMBA-treated Tgfbr1 cKO mice (FIG. 5B). Comparable elevated levels of the phosphorylated form of Akt (p-Akt) as well as the mammalian target of rapamycin (mTOR), a downstream target of the PI3K/Akt pathway, were observed in all of the tumors examined both by immunostaining and Western blot analysis (FIG. 5A, 5B). These results indicate that the PI3K/Akt pathway was activated in the SCCs that developed in the DMBA-treated Tgfbr1 cKO mice and that the loss of Tgfbr1 itself does not inactivate PTEN, therefore the reduced activity of PTEN may not be the leading cause to stimulate the PI3K/Akt pathway in the SCCs in DMBA-treated Tgfbr1 cKO mice.

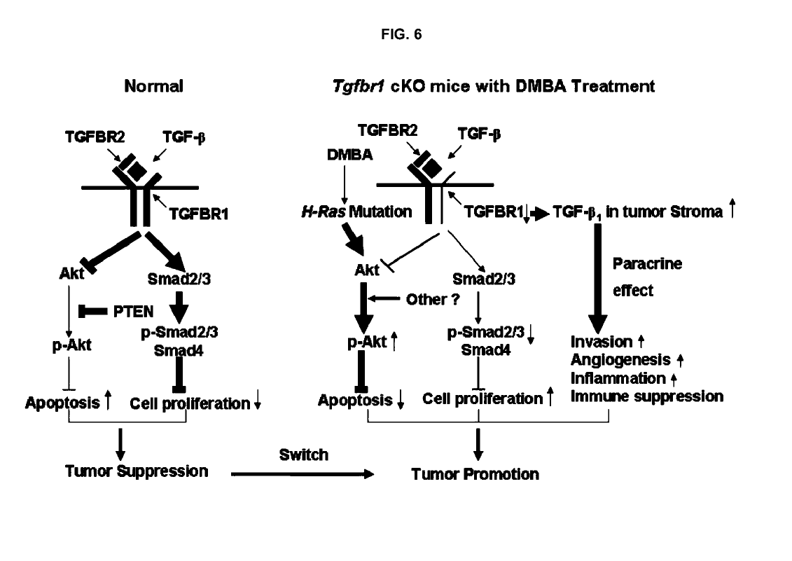

These results suggest that activation of Akt in the SCCs is perhaps independent of effects on PTEN in this mouse model and other mechanisms may be involved in the activation of this pathway, and one of these might be the H-ras mutations caused by DMBA initiation. Indeed, H-ras mutations were detected in 9 out of 17 tumors (53%) at codon 61 in exon 2 of the gene. However, the mechanisms underlying the activation of the PI3K/Akt pathway upon TGFβR1 deletion warrant further investigation. A proposed TGF-β signaling alteration that promotes SCC in mice through activation of PI3K/Akt pathway is shown in FIG. 6.

### Example 7

### Conditional Deletion of TGF-β Type I Receptor and PTEN Promotes Spontaneous SCC in Mice with Complete Penetrance

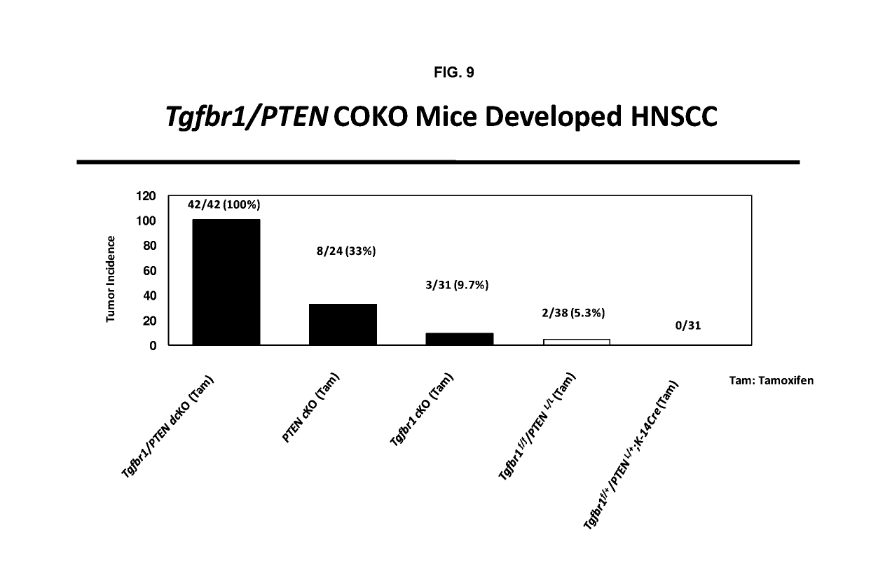

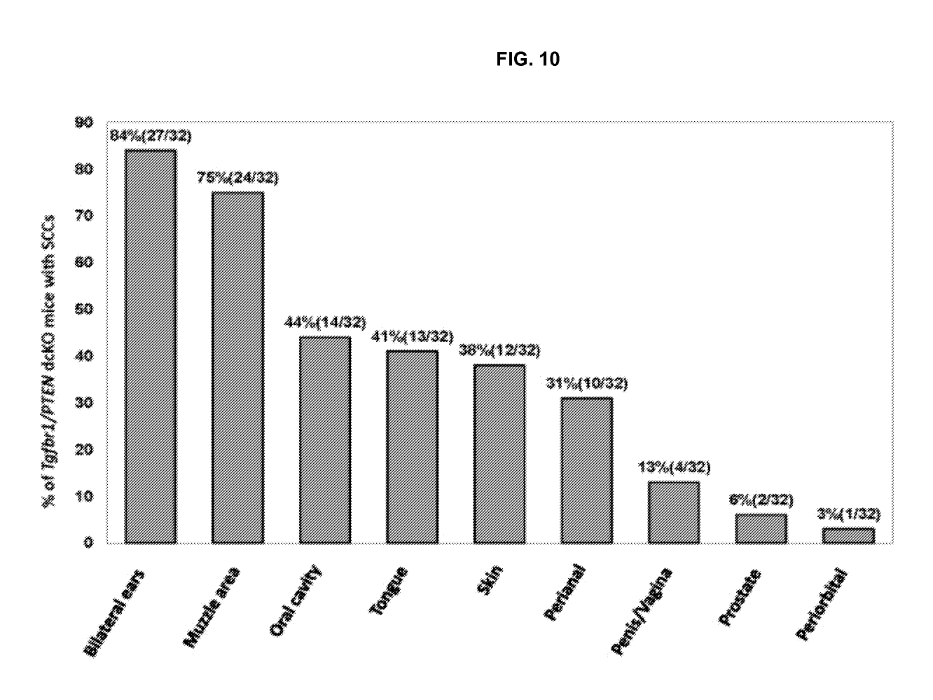

The results described above indicate that TGF-β suppresses head and neck carcinogenesis in cooperation with PI3K/Akt pathway. In order to further understand the molecular role of TGF-β type I receptor-mediated signaling and its cross talk with the PI3K/Akt pathway in the carcinogenesis of SCC, an inducible head- and neck-specific double knockout mouse model was generated by crossing Tgfbr1 floxed mice, PTEN loxp mice with K14-CreER^{tam} mice. By applying tamoxifen to the mouse oral cavity to induce Cre expression, conditional deletion of both Tgfbr1 and PTEN in the mouse head and neck epithelia was achieved. Loss of Tgfbr1 in combination with activation of PI3K/Akt due to PTEN deletion caused SCC with complete penetrance, while no tumors were observed in the control littermates (FIG. 9). Tgfbr1/PTEN cKO mice exhibited tumors in a number of different sites in the body (FIG. 10), including the ears (84%), muzzle (75%), oral cavity (44%), tongue (41%), skin (38%), perianal (31%), penis/vagina (13%), prostate (6%) and periorbital (3%).

A molecular analysis revealed an enhanced proliferation and loss of apoptosis in the basal layer of the head and neck epithelia of the Tgfbr1/PTEN cKO mice after tamoxifen treatment. In addition, an increase in inflammation, angiogenesis and myofibroblastic phenotype correlated with elevated levels of TGF-β1 were found in tumor stroma. These observations suggest that both autocrine and paracrine effects of TGF-β are involved in mouse head and neck carcinogenesis. Activation of Smad-independent pathways may contribute cooperatively with inactivation of Smad-dependent pathways to promote head and neck carcinogenesis in these mice. These findings have significant implications in developing effective therapeutic strategies targeting both the TGF-β and the PI3K/Akt pathways for the treatment of SCCs. These results also suggest that functional deficits due to mutations in Tgfbr1/PTEN are diagnostic of SCC or SCC susceptibility.

### Example 8

### TGFBR1 and PTEN Play a Role in Cancer Development in a Variety of Tissue Types

As described in Example 7, conditional deletion of TGFBR1 and PTEN was achieved by applying tamoxifen to the mouse oral cavity to induce Cre expression. This resulted in conditional deletion of these genes primarily in the oral mucosa. However, some of the tamoxifen applied in the oral mucosa leaked into the blood stream and/or spread to the paws and forelimbs due to the mice licking the applied tamoxifen and grooming the frontal areas. Thus, as shown in FIG. 10, tumors developed not only in the oral mucosa, but in several additional tissues. These results suggest that TGFBR1 and PTEN play a role in not only head and neck cancer, but in a variety of other types of cancer, including, for example, skin cancer, prostate cancer and cancers of the oral tissue, tongue, reproductive organs and peri-anal areas.

To further test the role of TGFBR1 and PTEN in other types of cancer, additional tissue-specific TGFBR1 and PTEN knockout animals can be generated using Cre transgenes regulated by tissue-specific promoters. For example, to evaluate the role of TGFBR1 and PTEN in liver cancer, transgenic mice expressing the albumin-Cre transgene can be used. Mice with the albumin-Cre transgene have been described (Postic et al., J. Biol. Chem. 274(1):305-315, 1999; Postic and Magnuson, Genesis 26(2):149-150, 2000) and are commercially available, such as from The Jackson Laboratory (Bar Harbor, Me.). Similarly, to evaluate cancer of the mammary glands, mice expressing the MMTV-Cre transgene can be used (described by Wagner et al., Nucleic Acids Res. 25(21):4323-4330, 1997; and Li et al., Development 129:4159-4170, 2002; and commercially available from The Jackson Laboratory). For prostate-specific expression, the probasin-Cre transgene (Maddison et al., Genesis 26(2):154-156, 2000; Wu et al., Mech. Dev. 101(1-2):61-69, 2001; available from the Mouse Models of Human Cancers Consortium, National Cancer Institute-Frederick), or the PSA-Cre transgene (Ma et al., Cancer Res. 65:5730-5739, 2005; available from The Jackson Laboratory) can be used.

Additional tissue-specific Cre transgenes are known in the art and can be used to induce deletion of TGFBR1 and PTEN. Moreover, tissue-specific promoters and methods of making tissue-specific Cre transgenes have been previously described and can be utilized to design a Cre transgene that is expressed in any desired type of tissue.

### Example 9

### Expression Levels of TGF-β and PTEN in HNSCC Cells

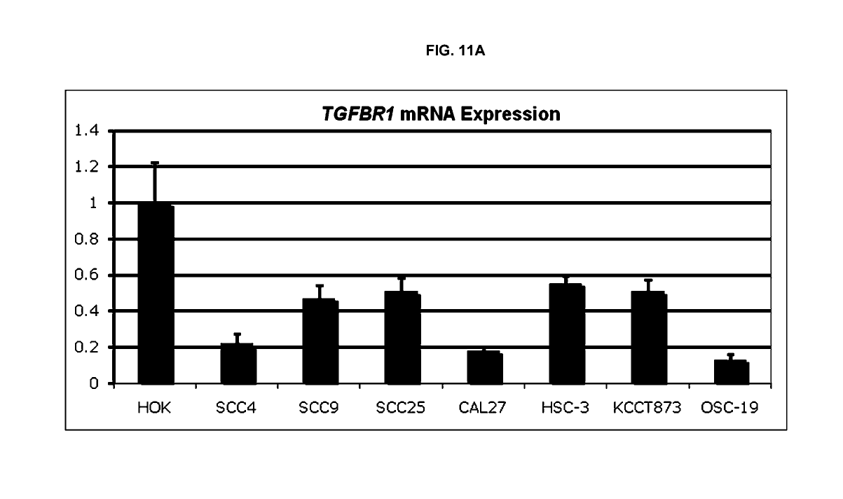

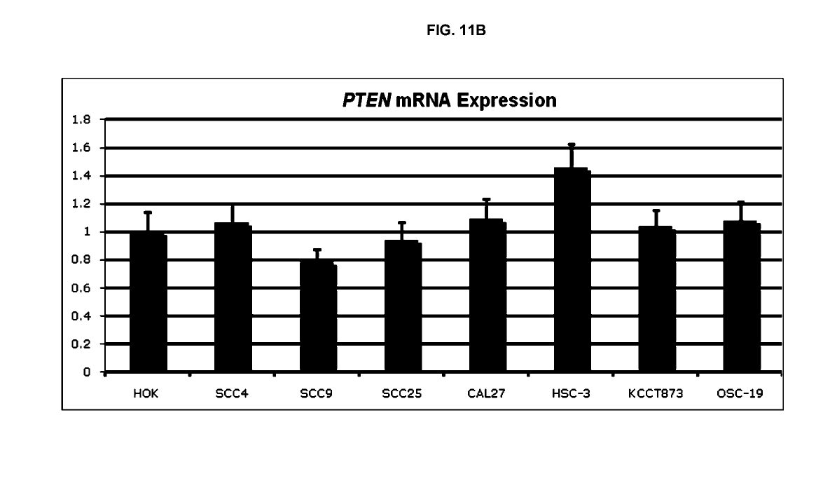

To determine whether defects in the TGF-β and PI3K/Akt signaling pathways often occur together in a subset of human squamous cell carcinomas, TGFBR1 and PTEN mRNA expression was examined in 7 human HNSCC cell lines (SCC4, SCC9, SCC25, CAL27, HSC-3, KCCT873 and OSC-19). The human oral keratinocyte (HOK) cell line (ScienceCell Research Laboratories, San Diego, Calif.) was used as a normal control. The qRT-PCR results revealed that the mRNA expression levels of TGFBR1 and PTEN were significantly reduced in 7/7 (100%) and 2/7 (29%) HNSCC cell lines, respectively (FIG. 11).

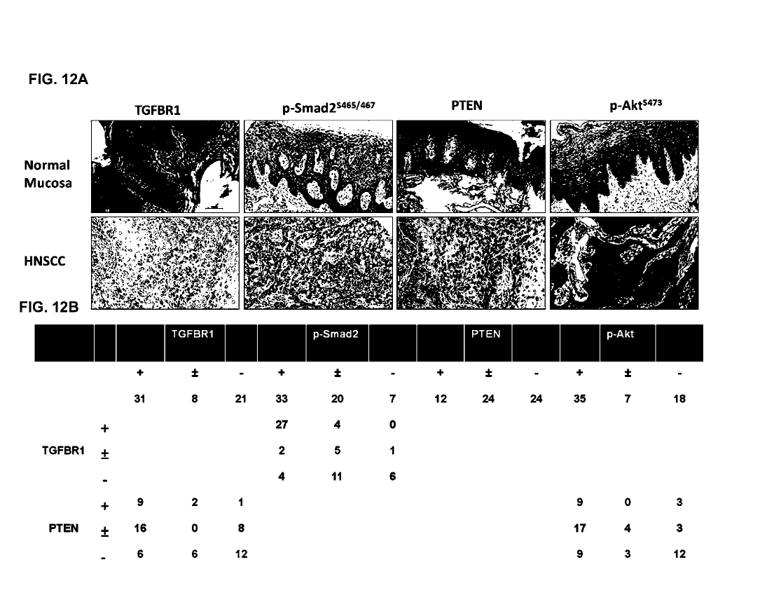

In addition, tissue array analysis was performed by immunostaining 60 human HNSCC samples and 12 normal controls. TGFBR1 and PTEN protein levels were found to be decreased in 29/60 (48%) and 48/60 (80%) HNSCC samples, respectively (FIG. 12). A similar decrease was also observed in phosphorylated Smad2, an activated mediator of TGF-β signaling ( 27/60, 45%). Also observed was an increase in p-Akt, a downstream target inhibited by PTEN ( 35/60, 58%). In total, 26 out of 60 HNSCC samples (43%) exhibited concurrent TGFBR1 and PTEN loss.

These results suggest that a significant proportion of human HNSCCs exhibit a reduction in expression of both TGFBR1 and PTEN, indicating that the TGFBR1/PTEN double conditional knockout mice are a relevant animal model for human cancer and that TGFBR1 and PTEN are useful as biomarkers for human cancer.

### Example 10

### Expression of IL-13Rα2 in TGFBR1 and PTEN cKO Tumors

A number of human cancers, such as head and neck cancer, glioblastoma, Kaposi's sarcoma, ovarian cancer and renal cell carcinoma, express high levels of IL-13 receptor (IL-13R), whereas normal cells or tissues derived from adjacent tissue exhibit little to no expression of IL-13R (Kawakami et al., J. Immunol. 169:7119-7126, 2002; Puri et al., Blood 87(10):4333-4339, 1996; Husain et al., Clin. Cancer Res. 3(2):151-156, 1997; Husain et al., Int J Cancer 92(2):168-175, 2001; Husain, et al., J Neuro-Oncol 65:37-48, 2003; Joshi et al., Clin. Cancer Res. 8:1948-1956, 2002). IL-13 binds to two receptor subunits, IL-13Rα1 and IL-13Rα2, and stimulates downstream signaling cascades involved in regulating cell proliferation and cell death in neoplastic cells. The IL-13Rα2 subunit binds IL-13 with high affinity and internalizes without the involvement of other chains. Thus, previous studies have investigated the use of an IL-13Rα2 targeted cytotoxin (IL13-PE38) for the treatment of cancer. IL13-PE38 is currently being studied in a Phase III clinical trial of brain cancer (Kawakami et al., J. Immunol. 169:7119-7126, 2002; Kioi et al., Int. J. Cancer 124:1440-1448, 2009).

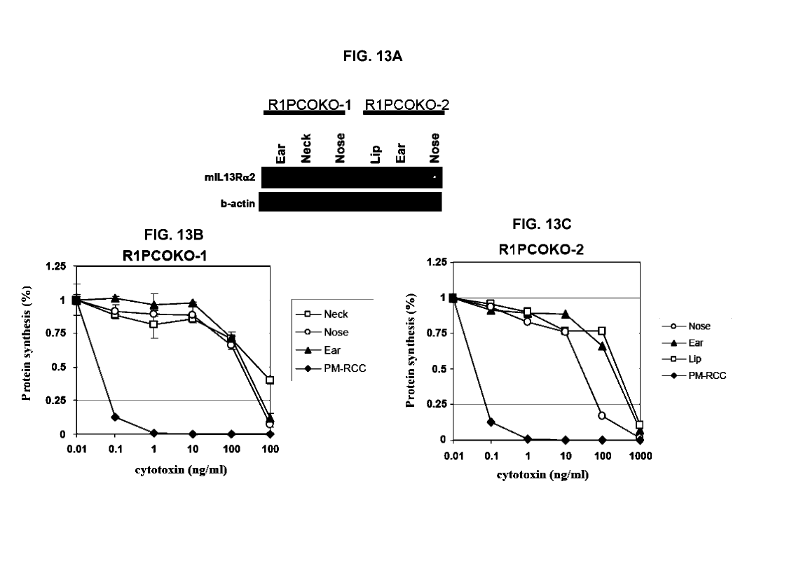

To determine whether primary cells derived from the spontaneous tumors that occur in all of the TGFBR1 and PTEN cKO mice express IL-13Rα2 mRNA, expression of IL-13Rα2 in primary cells derived from two TGFBR1 and PTEN cKO tumors was examined. The analysis revealed distinct expression of IL-13Rα2 mRNA in primary cells derived from the tumors localized in the ear, neck, nose and lips of these mice (FIG. 13). This observation is of particular importance because tumor cells from 30% of human HNSCC patients display similar expression of IL-13Rα2 mRNA.

It was further determined whether the primary cells from the mouse tumors are sensitive to the cytotoxin vector targeted against IL-13Rα2 (Honjo et al., Cell Cycle 6(11):1360-1366, 2007; U.S. Pat. No. 5,614,191). As shown in FIG. 13 (lower panels), cytotoxin treatment of the primary tumor cells from both the cKO mice resulted in significant reduction in protein synthesis similar to the human HNSCC cells (PM-RCC). This demonstrates that the tumors cells from the cKO mice display characteristics similar to human squamous cell carcinoma cells.

### Example 11

### Diagnosis and Treatment of a Subject with SCC

This example describes diagnosing a subject with SCC and specific examples of treating a subject diagnosed with SCC.

To select a subject in need of treatment, a patient undergoes a physical examination to detect any suspicious lesions (such as lesion in the head and neck, or on the skin). If a lesion (such as a tumor) is identified, a biopsy can be taken from the lesion to detect expression of TGFBR1 and PTEN mRNA or protein, or to detect the presence of tumor-associated mutations in TGFBR1 and PTEN. If the patient exhibits a decrease in expression of TGFBR1 and PTEN, or if at least one tumor-associated mutation is present in both TGFBR1 and PTEN, the subject is diagnosed with SCC, but additional diagnostic tests may be performed, for example prior to, concurrently or following the TGFBR1/PTEN analysis. The subject can further undergo additional diagnostic tests, such as histology of the biopsied lesion, additional genetic tests, x-ray, MRI or CAT scan.

Following diagnosis of SCC, an appropriate therapy is selected for the patient. Often, depending on the type and location of the lesion, surgical resection of the SCC tumor is performed. The subject may further be treated with radiation therapy, immunotherapy and/or chemotherapy. Alternatively, or in addition, the subject can be treated by administering an inhibitor of the PI3K/Akt pathway, a modulator of the TGF-β pathway, or both. In some cases, the modulator of the TGF-β pathway is an activator of the TGF-β pathway; in other cases, the modulator is an inhibitor of the TGF-β pathway. Non-limiting examples of PI3K/Akt pathway inhibitors TGF-β pathway modulators are described in the sections above.

### Example 12

### Treatment of a Patient with HNSCC

Following diagnosis of HNSCC in a subject, such as by using a method described in Example 11 above, an appropriate therapy is selected for the patient.

The three main types of treatment that have been used for managing HNSCC are radiation therapy, surgery and chemotherapy. Generally, the primary treatment is radiation therapy or surgery, or both. Typically, chemotherapy is used as an additional, or adjuvant, treatment, but may also be used as the primary treatment in some instances. The optimal combination of the three treatment modalities for a patient with HNSCC depends on the site of the cancer and the stage (extent) of the disease. Alternatively, or in addition to these treatments, the subject can be treated by administering an inhibitor of the PI3K/Akt pathway, a modulator of the TGF-β pathway, or both. In some cases, the modulator of the TGF-β pathway is an activator of the TGF-β pathway; in other cases, the modulator is an inhibitor of the TGF-β pathway. Non-limiting examples of PI3K/Akt pathway inhibitors TGF-β pathway modulators are described in the sections above.

In general, patients with early-stage HNSCC (particularly those limited to the site of origin) are treated with either radiation therapy or surgery. Patients who have more extensive cancers are often treated with concurrent chemotherapy and radiation therapy. Sometimes, depending on the clinical scenario, patients are treated with surgery followed by postoperative radiation therapy and/or chemotherapy. In each of these treatment plans, the patient may further be treated with an inhibitor of the PI3K/Akt pathway, a modulator of the TGF-β pathway, or both.

If the plan of treatment is radiation therapy for the primary cancer, the neck is generally also treated with radiation therapy. In addition, a neck dissection to remove involved lymph nodes in the neck may be necessary if the amount of disease in the neck nodes is relatively extensive or if the cancer in the neck nodes has not been eliminated completely by the end of the radiation therapy course.

Another treatment that might be necessary before or after radiation therapy is surgery. In general, if the surgical removal of the primary tumor is indicated, radiation is given afterward if necessary. Sometimes, however, the cancer is extensive or it is not feasible to completely remove the cancer initially. Radiotherapy is then given first to try to shrink the tumor, and surgery will follow radiotherapy.

Recent studies indicate that chemotherapy given at the same time as radiation therapy is more effective than if it is given before a course of radiation therapy. Therefore, radiation treatment schedules sometimes include chemotherapy if the stage of the cancer is advanced (advanced stage III or stage 1V). Drugs most commonly given in conjunction with radiation therapy are cisplatin (Platinol) and Cetuximab (Erbitux). Occasionally, other drugs may include fluorouracil (5-FU, Adrucil), carboplatin (Paraplatin), and paclitaxel (Taxol). This is only a partial list of chemotherapy agents; a physicians may choose to use others. The chemotherapy may be given in a variety of ways, including a low daily dose, a moderately low weekly dose, or a relatively higher dose every three to four weeks.

Typically, one of the following radiation therapy procedures may be used to treat HNSCC:

External beam therapy (EBT): a method for delivering a beam of high-energy x-rays to the location of the tumor. The beam is generated outside the patient (usually by a linear accelerator) and is targeted at the tumor site. These x-rays can destroy the cancer cells and careful treatment planning allows the surrounding normal tissues to be spared. No radioactive sources are placed inside the patient's body.

Intensity-modulated radiation therapy (IMRT): an advanced mode of high-precision radiotherapy that utilizes computer-controlled x-ray accelerators to deliver precise radiation doses to a malignant tumor or specific areas within the tumor. The radiation dose is designed to conform to the three-dimensional (3-D) shape of the tumor by modulating (or controlling) the intensity of the radiation beam to focus a higher radiation dose to the tumor while minimizing radiation exposure to healthy cells.

### Example 13

### Diagnosis and Treatment of a Patient with Prostate Cancer

This example describes diagnosing a subject with prostate cancer and specific examples of treating a subject diagnosed with prostate cancer.

To select a subject in need of treatment, a patient undergoes a physical examination to identify any suspicious lesions. If a lesion (such as a tumor) is identified, a biopsy can be taken from the lesion to detect expression of TGFBR1 and PTEN mRNA or protein, or to detect the presence of tumor-associated mutations in TGFBR1 and PTEN. If the patient exhibits a decrease in expression of TGFBR1 and PTEN, or if at least one tumor-associated mutation is present in both TGFBR1 and PTEN, the subject is diagnosed with prostate cancer, but additional diagnostic tests may be performed, for example prior to, concurrently or following the TGFBR1/PTEN analysis. The subject can further undergo additional diagnostic tests, such as histology of the biopsied lesion, additional genetic tests, x-ray, MRI or CAT scan.

Following diagnosis of prostate cancer, an appropriate therapy is selected for the patient. In some cases, the prostate cancer is treated by surgery (including prostatectomy). The subject may further be treated with radiation therapy (such as external beam radiation therapy or internal radiotherapy), immunotherapy, hormonal therapy and/or chemotherapy (such as temozolomide, doxorubicin, etoposide and/or paclitaxel). Alternatively, or in addition, the subject can be treated by administering an inhibitor of the PI3K/Akt pathway, a modulator of the TGF-β pathway, or both. In some cases, the modulator of the TGF-β pathway is an activator of the TGF-β pathway; in other cases, the modulator is an inhibitor of the TGF-β pathway. Non-limiting examples of PI3K/Akt pathway inhibitors TGF-β pathway modulators are described in the sections above.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method of diagnosing a subject as having squamous cell carcinoma (SCC), or being susceptible to developing SCC, comprising:
(i) detecting expression of transforming growth factor-β receptor type 1 (TGFBR1) and phosphatase and tensin homolog (PTEN) in a sample obtained from the subject; or
(ii) detecting the presence or absence of at least one tumor-associated mutation in the TGFBR1 gene and at least one tumor-associated mutation in the PTEN gene,
wherein a decrease in expression of TGFBR1 and PTEN in the sample; or the presence of the at least one mutation in TGFBR1 and the at least one mutation in PTEN in the sample, indicates the subject has SCC, or has increased susceptibility to developing SCC.

2. The method of claim 1, wherein the SCC is a SCC of the head and neck, skin, oral mucosa, tongue, peri-orbital region, penis, vagina, cervix or peri-anal region.

3. The method of claim 2, wherein the SCC is head and neck squamous cell carcinoma (HNSCC).

4. The method of claim 1, comprising detecting expression of TGFBR1 and PTEN in a sample obtained from the subject, wherein a decrease in expression of TGFBR1 and a decrease in expression of PTEN indicates the subject has SCC, or has increased susceptibility to developing SCC.

5. The method of claim 4, wherein detecting expression of TGFBR1 and PTEN in a sample comprises detecting the level of TGFBR1 and PTEN mRNA in the sample.

6. The method of claim 4, wherein detecting expression of TGFBR1 and PTEN in a sample comprises detecting the level of TGFBR1 and PTEN protein in the sample.

7-8. (canceled)

9. The method of claim 1, comprising detecting the presence or absence of at least one tumor-associated mutation in the TGFBR1 gene and at least one tumor-associated mutation in the PTEN gene, wherein the presence of the at least one mutation in TGFBR1 and the at least one mutation in PTEN indicates the subject has SCC, or has increased susceptibility to developing SCC.

10. The method of claim 9, wherein the at least one tumor-associated mutation in the TGFBR1 gene results in a decrease in expression of TGFBR1 mRNA or results in expression of a TGFBR1 protein with reduced activity, and wherein the at least one tumor-associated mutation in the PTEN gene results in a decrease in expression of PTEN mRNA or results in expression of a PTEN protein with reduced activity.

11. The method of claim 9, wherein the tumor-associated mutation in the TGFBR1 gene is (i) a complete or partial deletion of TGFBR1; (ii) TGFBR1 (6A); or (iii) TGFBR1 (10A).

12-13. (canceled)

14. The method of claim 9, wherein the tumor-associated mutation in the PTEN gene is a complete or partial deletion of PTEN; or a missense mutation in exon 5, 6, 7 or 8.

15. (canceled)

16. The method of claim 1, further comprising displaying the diagnostic results using an output device.

17-19. (canceled)

20. The method of claim 1, further comprising performing additional diagnostic tests to detect SCC if the method indicates the subject has SCC or increased susceptibility to SCC.

21. The method of claim 1, further comprising treating the subject for SCC.

22. The method of claim 21, wherein treating the subject for SCC comprises:
(i) administering a therapeutically effective amount of an inhibitor of the PI3K/Akt pathway;
(ii) administering a therapeutically effective amount of a modulator of the TGF-β pathway;
(iii) surgical removal of the SCC tumor;
(iv) administering radiation therapy;
(v) administering chemotherapy; or
(vi) any combination of two or more of (i) to (v).

23. A method of treating a subject with SCC, comprising: (i) selecting a subject in need of treatment; and (ii) administering to the subject a therapeutically effective amount of an inhibitor of the PI3K/Akt pathway and a therapeutically effective amount of a modulator of the TGF-β pathway, wherein administration of the inhibitor and modulator results in reduction in tumor size, inhibition of tumor growth, inhibition of tumor metastasis or inhibition of tumor progression, thereby treating the subject diagnosed with SCC.

24. The method of claim 23, wherein the SCC is a SCC of the head and neck, skin, penis, prostate, vagina or cervix.

25. The method of claim 24, wherein the SCC is HNSCC.

26. The method of claim 23, wherein the inhibitor of the PI3K/Akt pathway is an inhibitor of phosphoinositide-3 kinase (PI3K), AKT, pyruvate dehydrogenase kinase (PDK1) or mammalian target of rapamycin (mTOR).

27-29. (canceled)

30. The method of claim 23, wherein the modulator of the TGF-β pathway is an inhibitor of the TGF-β pathway.

31-33. (canceled)

34. The method of claim 23, wherein the modulator of the TGF-β pathway is an activator of the TGF-β pathway.

35-37. (canceled)

38. A pharmaceutical composition comprising an inhibitor of the PI3K/Akt pathway and a modulator of the TGF-β pathway.

39. The pharmaceutical composition of claim 38, wherein the modulator of the TGF-β pathway is an inhibitor of the TGF-β pathway.

40. The pharmaceutical composition of claim 38, wherein the modulator of the TGF-β pathway is an activator of the TGF-β pathway.

41-46. (canceled)

47. The pharmaceutical composition of claim 38, further comprising a pharmaceutically acceptable carrier.

48-49. (canceled)
